(19) United States
(12) Patent Application Publication
(10) Pub. No.: US 2024/0400642 A1
(43) Pub. Date: Dec. 5, 2024

(54) COMPOSITIONS AND METHODS FOR MODULATING MOLECULES

(71) Applicant: Etern Therapeutics Inc., New Castle, DE (US)

(72) Inventors: Guangya ZHU, Shanghai (CN); Jingjing XIE, Shanghai (CN); Yue YAN, Shanghai (CN); Jidong ZHU, Shanghai (CN)

(21) Appl. No.: 18/696,348

(22) PCT Filed: Jun. 26, 2023
(86) PCT No.: PCT/CN2023/102324

§ 371 Date: Mar. 27, 2024

(30) Foreign Application Priority Data
Jun. 27, 2022 (WO) PCT/CN2022/101584

(51) Int. Cl. C07K 14/705; A61K 31/7088; A61K 35/12; A61K 38/17; C07K 14/72; C07K 14/725; C12N 15/11

(52) U.S. Cl. CPC C07K 14/70567; A61K 31/7088; A61K 35/12; A61K 38/1783; A61K 38/1796; C07K 14/7051; C07K 14/721; C12N 15/11

(57) ABSTRACT

Provided are compositions for modulating molecules in a cell. Also provided are methods for modulating molecules in a cell.

### CROSS REFERENCE

This patent application claims priority to International Application No. PCT/CN2022/101584, filed Jun. 27, 2022, the entirety of which is hereby incorporated by reference herein.

### INCORPORATION BY REFERENCE

All publications, patents, and patent applications mentioned in this specification are herein incorporated by reference to the same extent as if each individual publication, patent, or patent application was specifically and individually indicated to be incorporated by reference. To the extent publications and patents or patent applications incorporated by reference contradict the disclosure contained in the specification, the specification is intended to supersede and or take precedence over any such contradictory material.

### BACKGROUND

Modulating molecules in a cell is critical for regulating cellular functions or maintaining overall cellular homeostasis or health. For example, molecules can be modulated (e.g., concentrated) in a specific location in a cell, where the molecules are brought in close proximity with one another, thus initiating enzymatic reaction or signaling cascade.

### SUMMARY

Accordingly, there is a need for compositions and methods for modulating molecules. Described herein, in some aspects, is an engineered polypeptide comprising: a functional domain; and a condensate shifting domain, said condensate shifting domain comprises an amino acid sequence of a nuclear receptor or fragment thereof. In some embodiments, the engineered polypeptide forms a first condensate within a cell by the engineered polypeptide operatively contacting with at least one additional condensate shifting domain. In some embodiments, the engineered polypeptide, upon contacted with a condensate modulator, forms a first condensate within a cell by the engineered polypeptide operatively contacting with at least one additional condensate shifting domain. In some embodiments, the first condensate mediates liquid-liquid phase separation (LLPS). In some embodiments, the first condensate comprises a liquid condensate. In some embodiments, the first condensate allows exchange of molecules between inside and outside of the first condensate. In some embodiments, the first condensate allows movement of molecules inside the first condensate. In some embodiments, the first condensate is reversed after removal of the condensate modulator. In some embodiments, the first condensate is reversed when the engineered polypeptide is contacted with a second condensate modulator. In some embodiments, the first condensate comprises a liquid condensate or a liquid-like condensate. In some embodiments, the first condensate is shifted to a second condensate when the engineered polypeptide is contacted with a third condensate modulator. In some embodiments, the second condensate is reversed back to the first condensate after removal of the third condensate modulator. In some embodiments, the second condensate is reversed back to the first condensate when the engineered polypeptide is contacted with a fourth condensate modulator. In some embodiments, the second condensate comprises a solid-like condensate or a semi-solid-like condensate. In some embodiments, the second condensate comprises a gel-like condensate. In some embodiments, the second condensate inhibits exchange of molecules between inside and outside of the second condensate. In some embodiments, the second condensate inhibits movement of molecules inside the second condensate. In some embodiments, the nuclear receptor or fragment thereof comprises an androgen receptor (AR) or a fragment thereof. In some embodiments, the nuclear receptor or fragment thereof comprises an amino acid sequence that is at least 70% identical to SEQ ID NO: 1. In some embodiments, the nuclear receptor or fragment thereof comprises an amino acid sequence that is at least 70% identical to SEQ ID NO: 2. In some embodiments, the nuclear receptor or fragment thereof comprises an amino acid sequence that is at least 70% identical to SEQ ID NO: 4. In some embodiments, the nuclear receptor or fragment thereof does not comprise an amino acid sequence that is at least 70% identical to SEQ ID NO: 3. In some embodiments, the nuclear receptor or fragment thereof comprises an estrogen receptor (ER) or a fragment thereof. In some embodiments, the nuclear receptor or fragment thereof comprises an amino acid sequence that is at least 70% identical to SEQ ID NO: 5. In some embodiments, the nuclear receptor or fragment thereof comprises an amino acid sequence that is at least 70% identical to SEQ ID NO: 6. In some embodiments, the nuclear receptor or fragment thereof comprises an amino acid sequence that is at least 70% identical to SEQ ID NO: 8. In some embodiments, the nuclear receptor or fragment thereof comprises an amino acid sequence that is at least 70% identical to SEQ ID NO: 7. In some embodiments, the condensate shifting domain comprises at least one modification. In some embodiments, the condensate shifting domain comprises at least one modification compared to any one of SEQ ID NOs: 1-10. In some embodiments, the at least one modification decreases binding between the engineered polypeptide and a nucleic acid by at least 70%, at least 80%, at least 90%, at least 95%, or at least 99% compared to binding between a comparable polypeptide without the at least one modification and the nucleic acid. In some embodiments, the at least one modification abolishes binding between the engineered polypeptide and a nucleic acid. In some embodiments, the at least one modification decreases binding between the engineered polypeptide and a ligand by at least 70%, at least 80%, at least 90%, at least 95%, or at least 99% compared to binding between a comparable polypeptide without the at least one modification and the ligand. In some embodiments, the at least one modification abolishes binding between the engineered polypeptide and the ligand. In some embodiments, the ligand is an endogenous ligand. In some embodiments, the ligand is an exogenous ligand. In some embodiments, the at least one modification comprises at least one truncation. In some embodiments, the at least one truncation comprises deletion of a nucleic acid binding domain or a fragment thereof. In some embodiments, the at least one modification comprises at least one amino acid substitution. In some embodiments, the at least one modification comprises at least one truncation and at least one amino acid substitution. In some embodiments, the at least one modification comprises A574D mutation or W742C mutation compared to SEQ ID NO: 1. In some embodiments, the at least one modification comprises A574D and W742C mutation compared to SEQ ID NO: 1. In some embodiments, the at least one modification comprises A574D mutation or W742L mutation compared to SEQ ID NO: 1. In some embodiments, the at least one modification comprises A574D mutation and W742L mutation compared to SEQ ID NO: 1. In some embodiments, the at least one modification comprises K632A mutation, K633A mutation, W742C mutation, or a combination thereof compared to SEQ ID NO: 1. In some embodiments, the at least one modification comprises K632A mutation, K633A mutation, and W742L mutation compared to SEQ ID NO: 1. In some embodiments, the at least one modification comprises truncation of amino acid residues 617-633 or W742C mutation compared to SEQ ID NO: 1. In some embodiments, the at least one modification comprises truncation of amino acid residues 617-633 and W742C mutation compared to SEQ ID NO: 1. In some embodiments, the at least one modification comprises truncation of amino acid residues 617-633 or W742L mutation compared to SEQ ID NO: 1. In some embodiments, the at least one modification comprises truncation of amino acid residues 617-633 and W742L mutation compared to SEQ ID NO: 1. In some embodiments, the at least one modification comprises truncation of amino acid residues 1-293; truncation of amino acid residues 556-666, W742C mutation, or a combination thereof compared to SEQ ID NO: 1. In some embodiments, the at least one modification comprises truncation of amino acid residues 1-293; truncation of amino acid residues 556-666, and W742C mutation compared to SEQ ID NO: 1. In some embodiments, the at least one modification comprises truncation of amino acid residues 1-293; truncation of amino acid residues 556-666, W742L mutation, or a combination thereof compared to SEQ ID NO: 1. In some embodiments, the at least one modification comprises truncation of amino acid residues 1-293; truncation of amino acid residues 556-666, and W742L mutation compared to SEQ ID NO: 1. In some embodiments, the at least one modification comprises truncation of amino acid residues 44-163; truncation of amino acid residues 226-347; truncation of amino acid residues 560-670, W742L mutation, or a combination thereof compared to SEQ ID NO: 1. In some embodiments, the at least one modification comprises truncation of amino acid residues 44-163; truncation of amino acid residues 226-347; truncation of amino acid residues 560-670, and W742L mutation compared to SEQ ID NO: 1. In some embodiments, the at least one medication comprises A574D mutation; W742C mutation; W742L mutation; K632A mutation; K633A mutation; truncation of amino acid residues 617-633; truncation of amino acid residues 44-163; truncation of amino acid residues 226-347; truncation of amino acid residues 560-670; truncation of amino acid residues 1-293; truncation of amino acid residues 556-666; or a combination thereof compared to SEQ ID NO: 1. In some embodiments, the at least one modification comprises K210A mutation, R211E mutation, L379R mutation, G521R mutation compared, or a combination thereof to SEQ ID NO: 5. In some embodiments, the at least one modification comprises K210A mutation, R211F mutation, 1.379R mutation, and G521R mutation compared to SEQ ID NO: 5. In some embodiments, the at least one modification comprises K210A, R211E, L379R, C400V, M543A, and L544A mutation compared to SEQ ID NO: 5. In some embodiments, the at least one modification comprises truncation of amino acid residues 250-274 compared to SEQ ID NO: 5. In some embodiments, the at least one modification comprises truncation of amino acid residues 250-274 or L379R mutation compared to SEQ ID NO: 5. In some embodiments, the at least one modification comprises truncation of amino acid residues 250-274 and L379R mutation compared to SEQ ID NO: 5. In some embodiments, the at least one modification comprises truncation of amino acid residues 250-274 or G521R mutation compared to SEQ ID NO: 5. In some embodiments, the at least one modification comprises truncation of amino acid residues 250-274 and G521R mutation compared to SEQ ID NO: 5. In some embodiments, the at least one modification comprises truncation of amino acid residues 250-274 and C400V, M543A, L544A mutation, or a combination thereof compared to SEQ ID NO: 5. In some embodiments, the at least one modification comprises truncation of amino acid residues 250-274 and C400V, M543A, and L544A mutation compared to SEQ ID NO: 5. In some embodiments, the at least one modification comprises K210A mutation; R211E mutation; L379R mutation; G521R mutation; C400V mutation; M543A mutation: L544A mutation; truncation of amino acid residues 250-274; or a combination thereof. In some embodiments, the at least one modification comprises truncation of amino acid residues 44-163; truncation of amino acid residues 226-347; truncation of amino acid residues 560-670, or a combination thereof compared to SEQ ID NO: 9. In some embodiments, the at least one modification comprises truncation of amino acid residues 44-163; truncation of amino acid residues 226-347; and truncation of amino acid residues 560-670 compared to SEQ ID NO: 9. In some embodiments, the at least one modification comprises truncation of amino acid residues 250-274 compared to SEQ ID NO: 10. In some embodiments, the condensate modulator comprises a functional domain ligand. In some embodiments, the functional domain ligand comprises a functional domain agonist. In some embodiments, the functional domain ligand comprises a functional domain antagonist. In some embodiments, the functional domain ligand comprises a functional domain inverse agonist. In some embodiments, the condensate modulator comprises a nuclear receptor ligand. In some embodiments, the condensate modulator comprises a nuclear receptor agonist, a nuclear receptor antagonist, or a nuclear receptor inverse agonist. In some embodiments, the condensate modulator comprises an androgen receptor agonist, an androgen receptor antagonist, or an androgen receptor inverse agonist. In some embodiments, the condensate modulator comprises an androgen receptor ligand comprising bicalutamide. In some embodiments, the condensate modulator comprises an estrogen receptor agonist, an estrogen receptor antagonist, or an estrogen receptor inverse agonist. In some embodiments, the condensate modulator comprises an estrogen receptor ligand comprising tamoxifen. In some embodiments, the second condensate modulator, the third condensate modulator, or the fourth condensate modulator comprises a nuclear receptor ligand. In some embodiments, the second condensate modulator, the third condensate modulator, or the fourth condensate modulator comprises a nuclear receptor agonist, a nuclear receptor antagonist, or a nuclear receptor inverse agonist. In some embodiments, the second condensate modulator, the third condensate modulator, or the fourth condensate modulator comprises an androgen receptor agonist, an androgen receptor antagonist, or an androgen receptor inverse agonist. In some embodiments, the second condensate modulator, the third condensate modulator, or the fourth condensate modulator comprises enzalutamide. In some embodiments, the second condensate modulator comprises enzalutamide. In some embodiments, the second condensate modulator, the third condensate modulator, or the fourth condensate modulator comprises an estrogen receptor agonist, an estrogen receptor antagonist, or an estrogen receptor inverse agonist. In some embodiments, the second condensate modulator, the third condensate modulator, or the fourth condensate modulator comprises fulvestrant. In some embodiments, the third condensate modulator comprises fulvestrant. In some embodiments, the engineered polypeptide forms the first condensate or the second condensate in cytoplasm of a cell. In some embodiments, the engineered polypeptide forms the first condensate or the second condensate in an organelle of a cell. In some embodiments, the engineered polypeptide forms the first condensate or the second condensate in nucleus of a cell. In some embodiments, the functional domain comprises a kinase domain. In some embodiments, the functional domain comprises a phosphatase domain. In some embodiments, the functional domain comprises a transactivation domain. In some embodiments, the functional domain comprises a DNA binding domain. In some embodiments, the functional domain comprises a metabolic enzyme domain. In some embodiments, the functional domain comprises a receptor. In some embodiments, the receptor comprises a CAR or fragment thereof. In some embodiments, the receptor comprises a TCR or fragment thereof.

Described herein, in some aspects, is an engineered polynucleotide encoding an engineered polypeptide described herein. In some embodiments, the engineered polynucleotide comprises a vector. In some embodiments, the vector comprises a viral vector. Described herein, in some aspects, is a cell comprising an engineered polypeptide or an engineered polynucleotide described herein.

Described herein, in some aspects, is a system comprising: an engineered polypeptide comprising a functional domain and a condensate shifting domain, said condensate shifting domain comprises a amino acid sequence of a nuclear receptor or fragment thereof; and a condensate modulator, where the engineered polypeptide, upon contacting with the condensate modulator, forms a first condensate comprising the engineered polypeptide operatively contacted with at least one other condensate shifting domain. In some embodiments, the system further comprises a second condensate modulator, a third condensate modulator, or a fourth condensate modulator.

Described herein, in some aspects, is a pharmaceutical composition comprising the engineered polypeptide, the engineered polynucleotide, the cell, or the system of any one of previous claims. In some embodiments, the pharmaceutical composition is formulated for administering intrathecally, intraocularly, intravitreally, retinally, intravenously, intramuscularly, intraventricularly, intracerebrally, intracerebellarly, intracerebroventricularly, intraperenchymally, subcutaneously, subretinally, suprachoroidally, intratumorally, pulmonarily, endotracheally, intraperitoneally, intravesically, intravaginally, intrarectally, orally, sublingually, transdermally, by inhalation, by inhaled nebulized form, by intraluminal-GI route, or a combination thereof to a subject in need thereof. In some embodiments, the pharmaceutical composition comprises at least one excipient. In some embodiments, the pharmaceutical composition comprises at least one additional active ingredient.

Described herein, in some aspects, is a method of forming a first condensate in a cell, said method comprising contacting the cell with the engineered polypeptide, the engineered polynucleotide, or the system of any one of previous claims, wherein the first condensate is formed when the engineered polypeptide is operatively contacted with at least one other condensate shifting domain. In some embodiments, the engineered polypeptide forms a first condensate within a cell by the engineered polypeptide operatively contacting with at least one additional condensate shifting domain. In some embodiments, the engineered polypeptide, upon contacted with a condensate modulator, forms a first condensate within a cell by the engineered polypeptide operatively contacting with at least one additional condensate shifting domain. In some embodiments, the first condensate mediates liquid-liquid phase separation (LLPS). In some embodiments, the first condensate comprises a liquid condensate. In some embodiments, the first condensate allows exchange of molecules between inside and outside of the first condensate. In some embodiments, the first condensate allows movement of molecules inside the first condensate. In some embodiments, the first condensate is reversed after removal of the condensate modulator. In some embodiments, the first condensate is reversed when the engineered polypeptide is contacted with a second condensate modulator. In some embodiments, the first condensate is shifted to a second condensate when the engineered polypeptide is contacted with a third condensate modulator. In some embodiments, the second condensate is reversed back to the first condensate after removal of the third condensate modulator. In some embodiments, the second condensate is reversed back to the first condensate when the engineered polypeptide is contacted with a fourth condensate modulator. In some embodiments, the second condensate comprises a solid-like condensate. In some embodiments, the second condensate comprises a semi-solid-like condensate. In some embodiments, the second condensate comprises a gel-like condensate. In some embodiments, the second condensate inhibits exchange of molecules between inside and outside of the second condensate. In some embodiments, the second condensate inhibits movement of molecules inside the second condensate.

Described herein, in some aspects, is a method for increasing enzymatic activity in a cell, said method comprising contacting the engineered polypeptide or the engineered polynucleotide of any one of previous claims with a condensate modulator for forming a first condensate when the engineered polypeptide is operatively contacted with at least one other condensate shifting domain, wherein the first condensate enrich local enzyme concentration. Also described herein, in some aspects, is a method for increasing enzymatic activity in a cell, said method comprising contacting the engineered polypeptide or the engineered polynucleotide of any one of previous claims with a condensate modulator for forming a first condensate when the engineered polypeptide is operatively contacted with at least one other condensate shifting domain, wherein the first condensate brings two or more molecules of the enzymatic activity to close proximity, thereby increases enzymatic activity. In some embodiments, the two or more molecules comprise at least one enzyme. In some embodiments, the two or more molecules comprise at least one biomolecule. In some embodiments, the two or more molecules comprise at least one nucleic acid. In some embodiments, the at least one nucleic acid comprises a DNA, a RNA, or a combination thereof. In some embodiments, the two or more molecules comprise at least one protein. In some embodiments, the at least one protein comprises a transcription factor. In some embodiments, the two or more molecules comprise at least one polysaccharide. In some embodiments, the two or more molecules comprises at least one lipid. In some embodiments, the two or more molecules comprise at least one metabolite.

Described herein, in some aspects, is a method for modulating concentration of at least one molecule in a cell, said method comprising contacting the cell with the engineered polypeptide or the engineered polynucleotide of any one of previous claims, wherein a first condensate is formed when the engineered polypeptide, upon contacted with a condensate modulator, is operatively contacted with at least one other condensate shifting domain, wherein a concentration of the at least one molecule inside the first condensate is different to a concentration of the at least one molecule outside the first condensate. In some embodiments, the concentration of the at least one molecule inside the first condensate is increased relatively to the concentration of the at least one molecule outside the first condensate. In some embodiments, the concentration is increased by at least 70%, at least 80%, at least 90%, at least 95%, or at least 99%. In some embodiments, the concentration of the at least one molecule inside the first condensate is decreased relatively to the concentration of the at least one molecule outside the first condensate. In some embodiments, the concentration is decreased by at least 70%, at least 80%, at least 90%, at least 95%, or at least 99%. In some embodiments, the first condensate is at least 10% more viscous, at least 20% more viscous, at least 30% more viscous, at least 40% more viscous, at least 50% more viscous, at least 60% more viscous, at least 70% more viscous, at least 80% more viscous, at least 90% more viscous, or at least 99% more viscous compared to a comparable first condensate formed by a comparable polypeptide without the at least one modification. In some embodiments, the first condensate is at least 10% less viscous, at least 20% less viscous, at least 30% less viscous, at least 40% less viscous, at least 50% less viscous, at least 60% less viscous, at least 70% less viscous, at least 80% less viscous, at least 90% less viscous, or at least 99% less viscous compared to a comparable first condensate formed by a comparable polypeptide without the at least one modification. In some embodiments, the first condensate comprises at least 10% more surface tension, at least 20% more surface tension, at least 30% more surface tension, at least 40% more surface tension, at least 50% more surface tension, at least 60% more surface tension, at least 70% more surface tension, at least 80% more surface tension, at least 90% more surface tension, or at least 99% more surface tension compared to a comparable first condensate formed by a comparable polypeptide without the at least one modification. In some embodiments, the first condensate comprises at least 10% less surface tension, at least 20% less surface tension, at least 30% less surface tension, at least 40% less surface tension, at least 50% less surface tension, at least 60% less surface tension, at least 70% less surface tension, at least 80% less surface tension, at least 90% less surface tension, or at least 99% less surface tension compared to a comparable first condensate formed by a comparable polypeptide without the at least one modification. In some embodiments, the first condensate comprises at least 10% more turbidity, at least 20% more turbidity, at least 30% more turbidity, at least 40% more turbidity, at least 50% more turbidity, at least 60% more turbidity, at least 70% more turbidity, at least 80% more turbidity, at least 90% more turbidity, or at least 99% more turbidity compared to a comparable first condensate formed by a comparable polypeptide without the at least one modification. In some embodiments, the first condensate comprises at least 10% less turbidity, at least 20% less turbidity, at least 30% less turbidity, at least 40% less turbidity, at least 50% less turbidity, at least 60% less turbidity, at least 70% less turbidity, at least 80% less turbidity, at least 90% less turbidity, or at least 99% less turbidity compared to a comparable first condensate formed by a comparable polypeptide without the at least one modification. In some embodiments, the first condensate increases a rate of exchange of molecule between inside and outside of the first condensate by at least 10%, at least 20%, at least 30%, at least 40%, at least 50%, at least 60%, at least 70%, at least 80%, at least 90%, or at least 99% compared to a rate of exchange of molecule between inside and outside of a comparable first condensate formed by a comparable polypeptide without the at least one modification. In some embodiments, the first condensate decreases a rate of exchange of molecule between inside and outside of the first condensate by at least 10%, at least 20%, at least 30%, at least 40%, at least 50%, at least 60%, at least 70%, at least 80%, at least 90%, or at least 99% compared to a rate of exchange of molecule between inside and outside of a comparable first condensate formed by a comparable polypeptide without the at least one modification. In some embodiments, the first condensate is at least 10%, at least 20%, at least 30%, at least 40%, at least 50%, at least 60%, at least 70%, at least 80%, at least 90%, or at least 99% more likely to accumulate in cytoplasm of a cell compared to a comparable first condensate formed by a comparable polypeptide without the at least one modification.

Described herein, in some aspects, is method for treating a disease or condition in a subject in need thereof, comprising administering the engineered polypeptide, the engineered polynucleotide, the cell, or the system of any one of previous claims to a subject. In some embodiments, the engineered polypeptide, the engineered polynucleotide, the cell, or the system of any one of previous claims forms a first condensate, a second condensate, a third condensate, or a combination thereof for treating the disease or condition in the subject.

### BRIEF DESCRIPTION OF THE DRAWINGS

This patent application contains at least one drawing executed in color. Copies of this patent or patent application with color drawing(s) will be provided by the Office upon request and payment of the necessary fec.

FIG. 1 illustrates an example of directing a signaling cascade in a cell by modulating (e.g., concentrating) molecules in a cell. By concentrating the molecules in the cell to a location, the signal cascade is driven towards predominantly D as opposed to the signal cascade split evenly between C and D due to the molecules not being modulated (e.g., passively diffused in the cell).

FIG. 2 illustrates examples of shifting between the different condensates in a cell meditated by the condensate shifting domain and the condensate modulator described herein.

FIG. 3A and FIG. 3B illustrates fluorescent images of condensate shifting in a cell. FIG. 3C and FIG. 3D illustrate western blot images of signaling cascade modulated by condensate forming or condensate shifting in a cell. FIG. 3A shows formation of a first condensate (where the first condensate is a phase inconsistent with its surrounding phase or medium), when the condensate shifting domain comprising an androgen receptor (AR) or fragment thereof was contacted with a condensate modulator comprising dihydrotestosterone (DHT). FIG. 3B shows formation of the first condensate, when the condensate shifting domain comprising an estrogen receptor (FR) or fragment thereof was contacted with a condensate modulator comprising bicalutamide (Bica). FIG. 3B further shows that the first condensate could be dissolved or reversed by contacting the condensate shifting domain comprising the AR or fragment thereof with an additional condensate modulator comprising enzalutamide (Enza). FIG. 3C shows western blot of protein phosphorylation regulated by the formation of first condensate, when the condensate shifting domain comprising an AR or ER or fragment thereof, was contacted with a condensate modulator comprising bicalutamide (Bica) or an additional condensate modulator comprising fulvestrant separately. FIG. 3D shows western blot result of protein phosphorylation regulated by the formation of first condensate, or by the shifting from first condensate to second condensate. The condensate shifting domain comprising an Androgen receptor (AR) or fragment thereof, was contacted with a condensate modulator comprising bicalutamide (Bica), or with a condensate modulator comprising bicalutamide (Bica) and an additional condensate modulator comprising enzalutamide (Enza) in succession.

The novel features of the disclosure are set forth with particularity in the appended claims. A better understanding of the features and advantages of the present disclosure will be obtained by reference to the following detailed description that sets forth illustrative embodiments.

### DETAILED DESCRIPTION

### Overview

Described herein, in some aspects, are compositions comprising an engineered polypeptide described herein for forming a first condensate in a cell. Also described herein are methods for forming the first condensate utilizing the engineered polypeptide described herein.

In some aspects, the first condensate is formed when the engineered polypeptide is contacted with a condensate modulator describe herein. In some embodiments, the first condensate can be formed intrinsically (e.g., without contacting the engineered polypeptide with a condensate modulator described herein). In some embodiments, the first condensate allows exchange of molecules between inside and outside of the first condensate. In some embodiments, the first condensate modulates concentration of the molecules, while allowing exchange of molecules between inside and outside of the first condensate. In some embodiments, the first condensate allows movement of molecules inside the first condensate. In some embodiments, the first condensate decreases exchange of at least one molecule described herein between inside and outside of the first condensate. In some embodiments, the first condensate inhibits exchange of at least one molecule described herein between inside and outside of the first condensate. In some embodiments, the first condensate decreases movement of at least one molecule described herein inside the first condensate. In some embodiments, the first condensate inhibits movement of at least one molecule described herein inside the first condensate. In some embodiments, the first condensate is reversed when the condensate modulator is removed. In some embodiments, the first condensate is reversed when the engineered polypeptide is contacted with a second condensate modulator described herein.

In some embodiments, the formation of the first condensate in the cell modulates molecules in the cell. For example, the first condensate can concentrate molecules in the first condensate and subsequently increase enzymatic activity by bringing molecules that are involved in an enzymatic activity in close proximity, thereby initiating the enzymatic activity. In some embodiments, the first condensate can modulate concentration of the molecules in the cell by sequestering the molecules in the first condensate, thereby decreasing the concentration of the molecules in the cell that is not part of the first condensate.

In some embodiments, the first condensate can be shifted to a second condensate when the engineered polypeptide is contacted with at least one additional condensate modulator (e.g., a third condensate modulator described herein). In some embodiments, the formation of the first condensate or second condensate in the cell can sequester the molecules in the first condensate or the second condensate, thereby reducing the concentration of the molecules in the cells that is not part of the condensate and decreasing enzymatic activity associated with the molecules sequestered by the first condensate or the second condensate. In some embodiments, the second condensate comprises a solid-like condensate, a semi-solid like condensate, a gel-like condensate, or a combination thereof. In some embodiments, the second condensate allows exchange of molecules between inside and outside of the second condensate. In some embodiments, the second condensate modulates concentration of the molecules, while allowing exchange of molecules between inside and outside of the second condensate. In some embodiments, the second condensate allows movement of molecules inside the second condensate. In some embodiments, the second condensate decreases exchange of at least one molecule described herein between inside and outside of the second condensate. In some embodiments, the second condensate inhibits exchange of at least one molecule described herein between inside and outside of the second condensate. In some embodiments, the second condensate decreases movement of at least one molecule described herein inside the second condensate. In some embodiments, the second condensate inhibits movement of at least one molecule described herein inside the second condensate. In some embodiments, the second condensate can be reversed by removing the third condensate modulator. In some embodiments, the second condensate can be reversed by contacting the engineered polypeptide with a fourth condensate modulator.

Described herein, in some aspects, is an engineered polypeptide comprising a functional domain and a condensate shifting domain, where the condensate shifting domain comprises an amino acid sequence of a nuclear receptor or fragment thereof. In some embodiments, the engineered polypeptide, upon contact with a condensate modulator, forms a first condensate within a cell by the engineered polypeptide operatively contacting with at least one additional condensate shifting domain (e.g., the engineered polypeptide operatively contacting with at least one additional condensate shifting domain from other engineered polypeptide molecules). In some embodiments, the first condensate is different from surrounding phase. For example, the first condensate can comprise different fluidity or viscosity comparing to a surrounding phase, where the surrounding phase is cytoplasm. In some embodiments, the first condensate mediates liquid-liquid condensate separation (LLPS). In some embodiments, the first condensate comprises a liquid condensate. In some embodiments, the first condensate comprises a liquid-like condensate.

In some embodiments, the first condensate modulates molecules in a cell by concentrating the molecules in the first condensate. In some embodiments, the first condensate allows exchange of molecules between inside and outside of the first condensate. In some embodiments, the first condensate modulates concentration of the molecules, while allowing exchange of molecules between inside and outside of the first condensate. In some embodiments, the first condensate allows movement of molecules inside the first condensate. In some embodiments, the first condensate, by modulating concentration of the molecules, can subsequently modulate enzymatic activity or signal cascade associated with the modulated molecules in the cell.

In some embodiments, the first condensate can be shifted to a second condensate by contacting the engineered polypeptide with at least one additional condensate modulator. In some embodiments, the second condensate, in contrast to the first condensate, does not allow movement of the molecules in the second condensate or the exchange of the molecules between inside and outside of the second condensate. In some embodiments, the second condensate, in contrast to the first condensate, decreases movement of the molecules in the second condensate or the exchange of the molecules between inside and outside of the second condensate. In some embodiments, the second condensate is a solid condensate, a semi-solid condensate, or a gel condensate. Solid condensate, semi-solid condensate, or gel condensate, in some aspects, comprises arbitrary shape and can have memory for retaining a shape over a period of time. In some aspects, the solid condensate, semi-solid condensate, or gel condensate comprises shear elasticity, which does not occur in the first condensate. In some embodiments, the second condensate comprises different fluidity or viscosity compared to the first condensate. In some embodiments, the second condensate inhibits enzymatic activity or signal cascade in the cell by arresting the molecules associated with the enzymatic activity or signal cascade in the second condensate. In some embodiments, the second condensate can be reversed to the first condensate when the engineered polypeptide is contacted with a fourth condensate modulator.

In some embodiments, the engineered polypeptide comprising the condensate shifting domain comprises an amino acid sequence that is at least 70% identical to an amino acid sequence of a nuclear receptor or fragment thereof. In some embodiments, the nuclear receptor or fragment thereof comprises an androgen receptor (AR) or a fragment thereof. In some embodiments, the nuclear receptor or fragment thereof comprises an estrogen receptor (ER) or a fragment thereof. In some embodiments, the condensate shifting domain comprising the amino acid sequence of the nuclear receptor or fragment thereof comprises at least one modification. In some embodiments, the at least one modification comprises at least one amino acid substitution. In some embodiments, the at least one modification comprises at least one truncation. In some embodiments, the at least one truncation comprises deletion of an entire domain such as a DNA binding domain (DBD) of a nuclear receptor described herein. In some embodiments, the at least one modification comprises at least one amino acid substitution and at least one truncation. In some embodiments, the at least one modification comprises at least one amino acid substitution and at least one truncation compared to a full length amino acid sequence of a nuclear receptor described herein. In some embodiments, the at least one modification comprises at least one amino acid substitution and at least one truncation compared to a full length amino acid sequence of an androgen receptor described herein. In some embodiments, the at least one modification comprises at least one amino acid substitution and at least one truncation compared to a full length amino acid sequence of an estrogen receptor described herein.

In some embodiments, the at least one modification decreases binding between the engineered polypeptide and a nucleic acid compared to binding between a comparable polypeptide without the at least one modification and the nucleic acid. In some embodiments, the at least one modification abolishes binding between the engineered polypeptide and a nucleic acid. In some embodiments, the at least one modification decreases binding between the engineered polypeptide and a ligand of the engineered polypeptide compared to binding between a comparable polypeptide without the at least one modification and the ligand. In some embodiments, the at least one modification abolishes binding between the engineered polypeptide and the ligand. The ligand for the engineered polypeptide can be either an endogenous ligand or an exogenous ligand. In some embodiments, the condensate modulator can be a functional domain ligand such as a functional domain agonist, a functional domain antagonist, or a functional domain inverse agonist.

In some embodiments, described herein is an engineered polynucleotide encoding an engineered polypeptide described herein. In some embodiments, the engineered polynucleotide comprises a vector such as a viral vector or a plasmid. In some embodiments, described herein is a cell comprising the engineered polypeptide or the engineered polynucleotide described herein. For example, the cell can be transduced with the engineered polynucleotide as part of a vector. In some embodiments, described herein is a system comprising: an engineered polypeptide comprising a functional domain and a condensate shifting domain, said condensate shifting domain comprises a amino acid sequence of a nuclear receptor or fragment thereof; and a condensate modulator, where the engineered polypeptide, upon contacting with the condensate modulator, forms a first condensate by the engineered polypeptide operatively contacted with at least one other condensate shifting domain. In some embodiments, the system further comprises at least one additional condensate modulator such as a second condensate modulate, a third condensate modulator, a fourth condensate modulator, or a combination thereof. In some embodiments, described herein is a pharmaceutical composition comprising the engineered polypeptide, the engineered polynucleotide, the cell, or the system described herein.

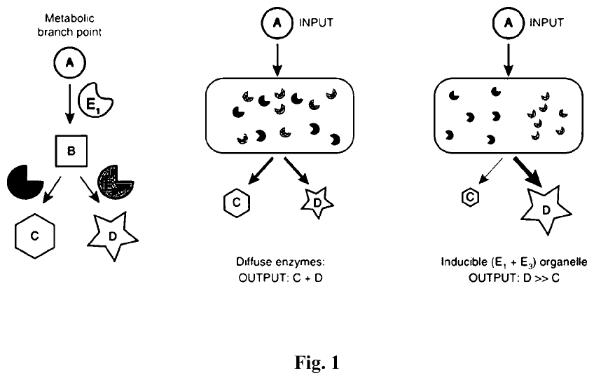

In some embodiments, described here is a method of forming a first condensate in a cell, said method comprising contacting the cell with the engineered polypeptide, the engineered polynucleotide, or the system, where the first condensate is formed when the engineered polypeptide, upon contacting with a condensate modulator, is operatively contacted with at least one other condensate shifting domain. In some embodiments, the method comprises shifting the first condensate to a second condensate when the engineered polypeptide is contacted with at least one additional condensate modulator. In some embodiments, the method comprises reversing the second condensate to the first condensate by contacting the engineered polypeptide with a third condensate modulator. Also described herein, in some aspects, is a method for increasing enzymatic activity in a cell, said method comprising contacting the engineered polypeptide with a condensate modulator for forming a first condensate when the engineered polypeptide is operatively contacted with at least one other condensate shifting domain, wherein the first condensate brings two or more molecules of the enzymatic activity to close proximity, thereby increases enzymatic activity. FIG. 1 illustrates an example of directing a signaling cascade in a cell by modulating (e.g., concentrating) molecules in a cell. By concentrating the molecules in the cell to a location, the signal cascade is driven towards predominantly D as opposed to the signal cascade split evenly between C and D due to the molecules not being modulated (e.g., passively diffused in the cell). In some aspects, the method described herein modulates concentration of at least one molecule in a cell. In some embodiments, the concentration of the at least one molecule inside the first condensate is increased relatively to the concentration of the at least one molecule outside the first condensate. In some embodiments, the concentration of the at least one molecule inside the first condensate is decreased relatively to the concentration of the at least one molecule outside the first condensate. In some embodiments, the compositions or methods can treat a disease or condition in a subject in need thereof. In some embodiments, described herein is a method for treating a disease or condition in a subject.

### Engineered Polypeptide

Described herein, in some embodiments, is an engineered polypeptide comprising a functional domain and a condensate shifting domain, said condensate shifting domain comprises an amino acid sequence of a nuclear receptor or fragment thereof. In some embodiments, the engineered polypeptide forms a first condensate when the engineered polypeptide is contacted with a condensate modulator described herein. The first condensate comprises a different phase compared to the surrounding phase. For example, the first condensate can comprise a liquid condensate that has different fluidity or viscosity compared to its surrounding phase. In some embodiments, the first condensate can be reversed by removing the condensate modulator. In some embodiments, the first condensate can be reversed by contacting the engineered polypeptide with at least one additional condensate modulator (e.g., a second condensate modulate described herein).

In some embodiments, the first condensate is shifted to a second condensate by contacting the engineered polypeptide with at least one additional condensate modulator (e.g., a third condensate modulator). In some embodiments, the second condensate is reversed back to the first condensate by removing the third condensate modulator. In some embodiments, the second condensate is reversed back to the first condensate by contacting the engineered polypeptide with a fourth condensate modulator.

In some embodiments, the engineered polypeptide can be introduced into a cell directly. In some embodiments, the engineered polypeptide can be delivered into the cell via an engineered polynucleotide described herein. In some embodiments, the formation of the first condensate can modulate enzymatic activity or signal cascade in a cell. In some embodiments, the formation of the first condensate or the second condensate can treat a disease or condition in a subject.

In some embodiments, the engineered polypeptide can be part of a system. For example, the engineered polypeptide is part of a system, where the system further comprises at least one condensate modulator described herein. In some embodiments, the engineered polypeptide, the engineered polynucleotide encoding the engineered polypeptide, or the system can be formulated into a pharmaceutical composition. In some embodiments, the engineered polypeptide, the engineered polynucleotide encoding the engineered polypeptide, or the system can be part of a kit.

### Functional Domain

In some embodiments, described herein is an engineered polypeptide comprising a functional domain. In some embodiments, the functional domain is operatively contacted to a condensate shifting domain described herein. In some embodiments, the functional domain is covalently connected to the condensate shifting domain. In some embodiments, the functional domain comprises a ligand binding domain. In some embodiments, the function domain can be contacted with a functional domain ligand, where the functional domain ligand is one of the condensate modulators describe herein. In some embodiments, the functional domain ligand comprises a functional domain agonist, a functional domain antagonist, or a functional domain inverse agonist. In some embodiments, the functional domain comprises a receptor. In some embodiments, the functional domain comprises an extracellular domain. In some embodiments, the functional domain comprises a transmembrane domain. In some embodiments, the functional domain comprises a cytoplasmic domain. In some embodiments, the functional domain comprises an extracellular domain, a transmembrane domain, a cytoplasmic domain, or a combination thereof.

In some embodiments, the functional domain comprises a kinase domain (e.g., a PKA domain or a PKC domain). In some embodiments, the functional domain comprises a phosphatase domain (e.g., a diacylglycerol pyrophosphate phosphatase, DGPP, domain). In some embodiments, the functional domain comprises a transactivation domain. Non-limiting example of the transactivation domain or the DNA binding domain comprises a domain of a transcription factor, a domain of a transcription coregulator, a domain of an epigenetic regulator, or a combination thereof. In some embodiments, the functional domain comprises a transactivation domain or a DNA binding domain. In some embodiments, the functional domain comprises a transactivation domain or a metabolic enzyme domain (e.g., domain of oxidoreductases, transferases, hydrolases, lyases, isomerases, ligases, or a combination thereof). In some embodiments, the functional domain comprises a receptor domain such as a chimeric antigen receptor (CAR) domain or a T cell receptor (TCR) domain. In some embodiments, the functional domain comprises a metabolism enzyme. In some embodiments, the CAR domain comprises at least a portion of a Notch receptor, a G-protein coupled receptor (GPCR), an integrin receptor, a cadherin receptor, a receptor tyrosine kinase, a death receptor, an immune receptor, or a combination thereof. In some embodiments, the immune receptor comprises a T cell receptor (TCR). In some embodiments, the receptor is endogenous to the cell. In some embodiments, the receptor is exogenous to the cell. Other example of the functional domain can include protein that is involved in TCR cluster, nephrin cluster, actin patch, focal adhesion, synaptic density, stress granule, RNA transport granule, U body, P body, Balbiani body, P granule, cGAS condensate, cleavage body, Cajal Body, GEM, nuclear speckle, OPT domain, PcG body, PML body, histone locus body, paraspeckle, or perinucleolar compartment. In some embodiments, the functional domain comprises gene-editing enzyme such as class 2 CRISPR/Cas, a modified version thereof, or a mutant version thereof. In some embodiments, the gene-editing enzyme comprises Cas9 or mutants of Cas9. In some embodiments, the gene-editing enzyme comprises SaCas9, CjCas9, SuperFi-Cas9, Cas12a, Cas14, CasΦ, or CasIscB.

### Condensate Shifting Domain

Described herein, in some aspects, is an engineered polypeptide comprising a condensate shifting domain. In some embodiments, the condensate shifting domain comprises an amino acid sequence that is at least 70%, at least 75%, at least 80%, at least 85%, at least 90%, at least 95%, at least 99%, or more identical to an amino acid sequence of a nuclear receptor or fragment thereof. For example, the condensate shifting domain comprises an amino acid sequence that is at least 70%, at least 75%, at least 80%, at least 85%, at least 90%, at least 95%, at least 99% or more identical to an amino acid sequence of SEQ ID NOs: 1-10 (Table 1). In some embodiments, the condensate shifting domain does not comprise a DNA binding domain (DBD). For example, the condensate shifting domain described herein, in some aspects, does not include the DBD encoded by SEQ ID NO: 3 or SEQ ID NO: 7.

TABLE 1
Exemplary amino acid sequence of the condensate shifting domain

| SEQ | | |
|---|---|---|
| ID | | |
| NO | Amino acid sequence | Note |
| SEQ | MEVQLGLGRVYPRPPSKTYRGAFQNLFQSVREVIQNPGPRHPEAASAAPPGASLLLLQQQQQQQQQ | Androgen |
| ID | QQQQQQQQQQQQQQQETSPRQQQQQGEDGSPQAHRRGPTGYLVLDEEQQPSQPQSALECHPERGCV | receptor |
| NO: | PEPGAAVAASKGLPQQLPAPPDEDDSAAPSTLSLLGPTFPGLSSCSADLKDILSEASTMQLLQQQQ | |
| 1 | QEAVSEGSSSGRAREASGAPTSSKDNYLGGTSTISDNAKELCKAVSVSMGLGVEALEHLSPGEQLR | |
| | GDCMYAPLLGVPPAVRPTPCAPLAECKGSLLDDSAGKSTEDTAEYSPFKGGYTKGLEGESLGCSGS | |
| | AAAGSSGTLELPSTLSLYKSGALDEAAAYQSRDYYNFPLALAGPPPPPPPPHPHARIKLENPLDYG | |
| | SAWAAAAAQCRYGDLASLHGAGAAGPGSGSPSAAASSSWHTLFTAEEGQLYGPCGGGGGGGGGGGG | |
| | GGGGGGGGGGGEAGAVAPYGYTRPPQGLAGQESDFTAPDVWYPGGMVSRVPYPSPTCVKSEMGPWM | |
| | DSYSGPYGDMRLETARDHVLPIDYYFPPQKTCLICGDEASGCHYGALTCGSCKVFFKRAAEGKQKY | |
| | LCASRNDCTIDKFRRKNCPSCRLRKCYEAGMTLGARKLKKLGNLKLQEEGEASSTTSPTEETTQKL | |
| | TVSHIEGYECQPIFLNVLEAIEPGVVCAGHDNNQPDSFAALLSSLNELGERQLVHVVKWAKALPGF | |
| | RNLHVDDQMAVIQYSWMGLMVFAMGWRSFTNVNSRMLYFAPDLVFNEYRMHKSRMYSQCVRMRHLS | |
| | QEFGWLQITPQEFLCMKALLLFSIIPVDGLKNQKFFDELRMNYIKELDRIIACKRKNPTSCSRRFY | |
| | QLTKLLDSVQPIARELHQFTFDLLIKSHMVSVDFPEMMAEIISVQVPKILSGKVKPIYFHTQ | |
| SEQ | ECQPIFLNVLEAIEPGVVCA GHDNNQPDSFAALLSSLNEL GERQLVHVVKWAKALPGERN | AR |
| ID | LHVDDQMAVIQYSWMGLMVF AMGWRSFTNVNSRMLYFAPD LVFNEYRMHKSRMYSQCVRM | ligand |
| NO: | RHLSQEFGWLQITPQEFLCM KALLLFSIIPVDGLKNQKFF DELRMNYIKELDRIIACKRK | binding |
| 2 | NPTSCSRRFYQLTKLLDSVQ PIARELHQFTFDLLIKSHMV SVDFPEMMAEIISVQVPKIL | domain |
| | SGKVKPIYFHTQ | (LBD) |
| SEQ | CLICGDEASGCHYGALTCGSCKVFFKRAAEGKQKYLCASRNDCTIDKFRRKNCPSCRLRKCYEAGM | AR DNA |
| ID | TLGARKLKKL | binding |
| NO: | | domain |
| 3 | | (DBD) |
| SEQ | MEVQLGLGRVYPRPPSKTYRGAFQNLFQSVREVIQNPGPRHPEAASAAPPGASLLLLQQQQQQQQQ | Truncated |
| ID | QQQQQQQQQQQQQQQETSPRQQQQQGEDGSPQAHRRGPTGYLVLDEEQQPSQPQSALECHPERGCV | AR |
| NO: | PEPGAAVAASKGLPQQLPAPPDEDDSAAPSTLSLLGPTFPGLSSCSADLKDILSEASTMQLLQQQQ | (DNA |
| 4 | QEAVSEGSSSGRAREASGAPTSSKDNYLGGTSTISDNAKELCKAVSVSMGLGVEALEHLSPGEQLR | binding |
| | GDCMYAPLLGVPPAVRPTPCAPLAECKGSLLDDSAGKSTEDTAEYSPFKGGYTKGLEGESLGCSGS | domain |
| | AAAGSSGTLELPSTLSLYKSGALDEAAAYQSRDYYNFPLALAGPPPPPPPPHPHARIKLENPLDYG | is |
| | SAWAAAAAQCRYGDLASLHGAGAAGPGSGSPSAAASSSWHTLFTAEEGQLYGPCGGGGGGGGGGGG | removed) |
| | GGGGGGGGGGGEAGAVAPYGYTRPPQGLAGQESDFTAPDVWYPGGMVSRVPYPSPTCVKSEMGPWM | |
| | DSYSGPYGDMRLETARDHVLPIDYYFPPQKTGNLKLQEEGEASSTTSPTEETTQKLTVSHIEGYEC | |
| | QPIFLNVLEAIEPGVVCAGHDNNQPDSFAALLSSLNELGERQLVHVVKWAKALPGERNLHVDDQMA | |
| | VIQYSWMGLMVFAMGWRSFTNVNSRMLYFAPDLVENEYRMHKSRMYSQCVRMRHLSQEFGWLQITP | |
| | QEFLCMKALLLFSIIPVDGLKNQKFFDELRMNYIKELDRIIACKRKNPTSCSRRFYQLTKLLDSVQ | |
| | PIARELHQFTFDLLIKSHMVSVDFPEMMAEIISVQVPKILSGKVKPIYFHTQ | |
| SEQ | MTMTLHTKASGMALLHQIQGNELEPLNRPQLKIPLERPLGEVYLDSSKPAVYNYPEGAAYEFNAAA | Estrogen |
| ID | AANAQVYGQTGLPYGPGSEAAAFGSNGLGGFPPLNSVSPSPLMLLHPPPQLSPFLQPHGQQVPYYL | receptor |
| NO: | ENEPSGYTVREAGPPAFYRPNSDNRRQGGRERLASTNDKGSMAMESAKETRYCAVCNDYASGYHYG | |
| 5 | VWSCEGCKAFFKRSIQGHNDYMCPATNQCTIDKNRRKSCQACRLRKCYEVGMMKGGIRKDRRGGRM | |
| | LKHKRQRDDGEGRGEVGSAGDMRAANLWPSPLMIKRSKKNSLALSLTADQMVSALLDAEPPILYSE | |
| | YDPTRPFSEASMMGLLTNLADRELVHMINWAKRVPGFVDLTLHDQVHLLECAWLEILMIGLVWRSM | |
| | EHPGKLLFAPNLLLDRNQGKCVEGMVEIFDMLLATSSRFRMMNLQGEEFVCLKSIILLNSGVYTFL | |
| | SSTLKSLEEKDHIHRVLDKITDTLIHLMAKAGLTLQQQHQRLAQLLLILSHIRHMSNKGMEHLYSM | |
| | KCKNVVPLYDLLLEMLDAHRLHAPTSRGGASVEETDQSHLATAGSTSSHSLQKYYITGEAEGFPAT | |
| | V | |
| SEQ | TADQMVSALLDAEPPILYSEYDPTRPFSEASMMGLLTNLADRELVHMINWAKRVPGFVDLTLHDQV | ER |
| ID | HLLECAWLEILMIGLVWRSMEHPGKLLFAPNLLLDRNQGKCVEGMVEIFDMLLATSSRFRMMNLQG | ligand |
| NO: | EEFVCLKSIILLNSGVYTFLSSTLKSLEEKDHIHRVLDKITDTLIHLMAKAGLTLQQQHQRLAQLL | binding |
| 6 | LILSHIRHMSNKGMEHLYSMKCKNVVPLYDLLLEMLDAH | domain |
| | | (LBD) |
| SEQ | KETRYCAVCNDYASGYHYGVWSCEGCKAFFKRSIQGHNDYMCPATNQCTIDKNRRKSCQACRLRKC | ER |
| ID | YEVGM | DNA |
| NO: | | binding |
| 7 | | domain |
| | | (DBD) |
| SEQ | MTMTLHTKASGMALLHQIQGNELEPLNRPQLKIPLERPLGEVYLDSSKPAVYNYPEGAAYEFNAAA | Truncated |
| ID | AANAQVYGQTGLPYGPGSEAAAFGSNGLGGFPPLNSVSPSPLMLLHPPPQLSPFLQPHGQQVPYYL | ER |
| NO: | ENEPSGYTVREAGPPAFYRPNSDNRRQGGRERLASTNDKGSMAMESAMKGGIRKDRRGGRMLKHKR | (DNA |
| 8 | QRDDGEGRGEVGSAGDMRAANLWPSPLMIKRSKKNSLALSLTADQMVSALLDAEPPILYSEYDPTR | binding |
| | PFSEASMMGLLTNLADRELVHMINWAKRVPGFVDLTLHDQVHLLECAWLEILMIGLVWRSMEHPGK | domain |
| | LLFAPNLLLDRNQGKCVEGMVEIFDMLLATSSRFRMMNLQGEEFVCLKSIILLNSGVYTFLSSTLK | is |
| | SLEEKDHIHRVLDKITDTLIHLMAKAGLTLQQQHQRLAQLLLILSHIRHMSNKGMEHLYSMKCKNV | removed) |
| | VPLYDLLLEMLDAHRLHAPTSRGGASVEETDQSHLATAGSTSSHSLQKYYITGEAEGFPAT | |
| SEQ | MEVQLGLGRVYPRPPSKTYRGAFQNLFQSVREVIQNPGPRHPEAASAAPPGASLLLLQQQQQQQQQ | Truncated |
| ID | QQQQQQQQQQQQQQETSPRQQQQQQGEDGSPQAHRRGPTGYLVLDEEQQPSQPQSALECHPERGCV | AR |
| NO: | PEPGAAVAASKGLPQQLPAPPDEDDSAAPSTLSLLGPTFPGLSSCSADLKDILSEASTMQLLQQQQ | (Ligand |
| 9 | QEAVSEGSSSGRAREASGAPTSSKDNYLGGTSTISDNAKELCKAVSVSMGLGVEALEHLSPGEQLR | binding |
| | GDCMYAPLLGVPPAVRPTPCAPLAECKGSLLDDSAGKSTEDTAEYSPFKGGYTKGLEGESLGCSGS | domain |
| | AAAGSSGTLELPSTLSLYKSGALDEAAAYQSRDYYNFPLALAGPPPPPPPPHPHARIKLENPLDYG | is |
| | SAWAAAAAQCRYGDLASLHGAGAAGPGSGSPSAAASSSWHTLFTAEEGQLYGPCGGGGGGGGGGGG | removed) |
| | GGGGGGGGGGGEAGAVAPYGYTRPPQGLAGQESDFTAPDVWYPGGMVSRVPYPSPTCVKSEMGPWM | |
| | DSYSGPYGDMRLETARDHVLPIDYYFPPQKTCLICGDEASGCHYGALTCGSCKVFFKRAAEGKQKY | |
| | LCASRNDCTIDKFRRKNCPSCRLRKCYEAGMTLGARKLKKLGNLKLQEEGEASSTTSPTEETTQKL | |
| | TVSHIEGYEC | |
| SEQ | MTMTLHTKASGMALLHQIQGNELEPLNRPQLKIPLERPLGEVYLDSSKPAVYNYPEGAAYEFNAAA | Truncated |
| ID | AANAQVYGQTGLPYGPGSEAAAFGSNGLGGFPPLNSVSPSPLMLLHPPPQLSPFLQPHGQQVPYYL | ER |
| NO: | ENEPSGYTVREAGPPAFYRPNSDNRRQGGRERLASTNDKGSMAMESAKETRYCAVCNDYASGYHYG | (Ligand |
| 10 | VWSCEGCKAFFKRSIQGHNDYMCPATNQCTIDKNRRKSCQACRLRKCYEVGMMKGGIRKDRRGGRM | binding |
| | LKHKRQRDDGEGRGEVGSAGDMRAANLWPSPLMI | domain |
| | | is |
| | | removed) |

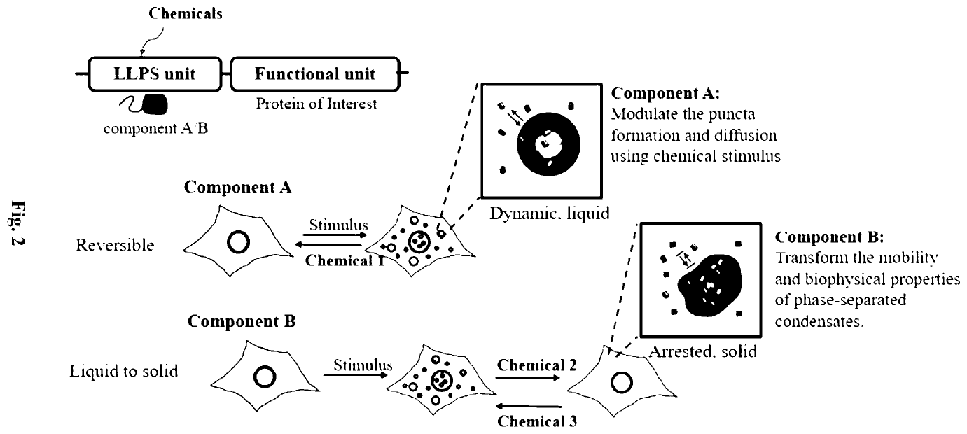

In some embodiments, the condensate shifting domain comprises at least one modification compared to a wild type amino acid sequence of a nuclear receptor or fragment thereof. In some embodiments, the nuclear receptor or fragment thereof comprises androgen receptor (AR) or fragment thereof. In some embodiments, the nuclear receptor or fragment thereof comprises estrogen receptor (ER) or fragment thereof. In some embodiments, the at least one modification decreases binding between the engineered polypeptide and a nucleic acid by at least 70%, at least 80%, at least 90%, at least 95%, or at least 99% compared to binding between a comparable polypeptide without the at least one modification and the nucleic acid. In some embodiments, the at least one modification abolishes binding between the engineered polypeptide and a nucleic acid. In some embodiments, the at least one modification comprises deletion of a DBD from the condensate shifting domain, where the condensate shifting domain can no longer bind to the nucleic acid. In some embodiments, the at least one modification decreases binding between the engineered polypeptide and a ligand by at least 70%, at least 80%, at least 90%, at least 95%, or at least 99% compared to binding between a comparable polypeptide without the at least one modification and the ligand. In some embodiments, the at least one modification abolishes binding between the engineered polypeptide and the ligand. In some embodiments, the ligand is an endogenous ligand. In some embodiments, the ligand is an exogenous ligand. In some embodiments, the at least one modification increases the formation of the first condensate or the second condensate by increasing the binding affinity between the condensate shifting domain with at least one other condensate shifting domain. FIG. 2 illustrates examples of shifting between the different phases in a cell meditated by the condensate shifting domain and the condensate modulator described herein.

### Androgen Receptor

In some embodiments, the condensate shifting domain described herein comprises an amino acid sequence of androgen receptor (AR) or fragment thereof. AR is a member of the steroid and nuclear receptor superfamily and is mainly expressed in androgen target tissues, such as the prostate, skeletal muscle, liver, and central nervous system (CNS), with the highest expression level observed in the prostate, adrenal gland, and epididymis. AR is a soluble protein that functions as an intracellular transcriptional factor. Upon binding and activation by androgens such as testosterone or dihydrotestosterone (DHT), AR undergoes conformational changes and posttranslational modifications, dimerization, nuclear translocation, and ultimately, binding to the regulatory regions of the DNA of target genes, known as androgen response elements.

Wild type AR contains three distinct domains, including an androgen-independent N-terminal domain (NTD), a DNA binding domain (DBD), and an androgen-dependent ligand-binding domain (LBD). The full length wild type AR has an amino acid sequence of SEQ ID NO: 1, and NTD in the full length wild type AR spans from the first 559 amino acid residues. NTD of AR contains high degree of intrinsic disorder, with few alpha-helices and beta-sheets. The disordered region that lack a fixed or ordered secondary and tertiary structure in a protein is also referred to as intrinsic disordered regions (IDRs). IDRs can range from fully unstructured to partially structured. NTD of AR is found to contain extensive IDRs. Without bound to any theory, phase separation or condensate formation of AR is believed to be driven by multivalent interaction via adhesions or associations mediated by IDR. In addition, NTD of AR is known to contain the activation function-1 (AF1) region, which is essential for AR transactivation and is present in different forms of the AR variants. The term “androgen receptor” or “AR” as used herein encompasses both wild type AR or AR variant (e.g., AR comprising at least one modification). Wild type AR as used herein refers to the full length AR, having an exemplary sequence of SEQ ID NO: 1. AR variant as used herein encompasses all different forms of AR that retains at least part of the NTD and can phase separate or can form condensates. Examples of AR variants include, without limitation, mutants, fragments, fusions, and splicing variants. In some embodiments, the AR variant is resistant to at least one androgen-deprivation therapy. The term “androgen deprivation therapy” or “ADT” as used herein refers to therapies that suppress androgen, by reducing levels of androgen or by inhibiting biological functions of androgen such as by inhibiting AR signaling. ADT can include both surgical treatments (such as surgical castration) and drug treatments. Examples of ADT drugs include, without limitation, LHRH agonists such as Leuprolide (Lupron, Eligard), Goserelin (Zoladex), Triptorelin (Trelstar), and Histrelin (Vantas); LHRH antagonists such as Degarelix (Firmagon) and Relugolix (Orgovyx); drugs that lower androgen levels from the adrenal glands such as Abiraterone (Zytiga) and Ketoconazole (Nizoral); androgen receptor antagonists such as Flutamide (Eulexin). Bicalutamide (Casodex), and Nilutamide (Nilandron); and other anti-androgens such as Enzalutamide (Xtandi), apalutamide (Erleada), and darolutamide (Nubeqa).

In some embodiments, the AR variant comprises one or more mutations. Over 800 different mutations have been identified with AR in patients with androgen insensitivity syndrome, and prostate cancer. In the AR gene, four different types of mutations have been detected to inactivate AR, including: single point mutations resulting in amino acid substitutions or premature stop codons; nucleotide insertions or deletions leading to a frame shift and premature rumination; complete or partial gene deletions; and intronic mutations causing alternative splicing). In some embodiments, the AR variant comprises one or more mutations in ligand binding domain (LBD). In some embodiments, the one or more mutations in the LBD results in gain of function in AR. In some embodiments, the AR variant comprises one or more mutations at the residues selected from the group consisting of A574, K632. K633, L702, V716, V731. W742, H875, F877, T878, D880, L882, S889, D891, E894, M896, E898, and T919, wherein the numbering is relative to SEQ ID NO: 1. In some embodiments, the one or more mutations are selected from the group consisting of A574D, K632A, K633A, L702H, V716M, V731M, W742L/C, H875Y/Q, F877L, T878A/S, D880E, L882I, S889G, D891H, E894K, M896V/T, F898G, and T919S.

In some embodiments, the AR variant lacks all or part of DBD. For example, the AR variant lacks DBD encoded by SEQ ID NO: 3. In some embodiments, the AR variant lacks all or part of LBD comprises an amino acid sequence that is at least 70%, at least 80%, at least 90%, at least 95%, or at least 99% identical to SEQ ID NO: 4. In some embodiments, the condensate shifting domain comprises at least one modification comprising the at least one modification comprises A574D mutation or W742C mutation compared to SEQ ID NO: 1. In some embodiments, the condensate shifting domain comprises at least one modification comprising the at least one modification comprises A574D mutation and W742C mutation compared to SEQ ID NO: 1. In some embodiments, the condensate shifting domain comprises at least one modification comprising A574D mutation or W742L mutation compared to SEQ ID NO: 1. In some embodiments, the condensate shifting domain comprises at least one modification comprising A574D mutation and W742L mutation compared to SEQ ID NO: 1. In some embodiments, the condiment shifting domain comprises at least one modification comprises K632A mutation; K633A mutation; W742C mutation; or a combination thereof compared to SEQ ID NO: 1. In some embodiments, the condensate shifting domain comprises at least one modification comprising K632A mutation, K633A mutation, and W742C mutation compared to SEQ ID NO: 1. In some embodiments, the condensate shifting domain comprises at least one modification comprising K632A mutation, K633A mutation, W742L mutation, or a combination thereof compared to SEQ ID NO: 1. In some embodiments, the condensate shifting domain comprises at least one modification comprising K632A mutation, K633A mutation, and W742L mutation compared to SEQ ID NO: 1. In some embodiments, the condensate shifting domain comprises at least one modification comprising a truncation of amino acid residues 617-633 or W742C mutation compared to SEQ ID NO: 1. In some embodiments, the condensate shifting domain comprises at least one modification comprising a truncation of amino acid residues 617-633 and W742C mutation compared to SEQ ID NO: 1. In some embodiments, the condensate shifting domain comprises at least one modification comprising a truncation of amino acid residues 617-633 or W742L mutation compared to SEQ ID NO: 1. In some embodiments, the condensate shifting domain comprises at least one modification comprising a truncation of amino acid residues 617-633 and W742L mutation compared to SEQ ID NO: 1. In some embodiments, the condensate shifting domain comprises at least one modification comprising a truncation of amino acid residues 1-293; truncation of amino acid residues 556-666, W742C mutation, or a combination thereof compared to SEQ ID NO: 1. In some embodiments, the condensate shifting domain comprises at least one modification comprising a truncation of amino acid residues 1-293; truncation of amino acid residues 556-666, and W742C mutation compared to SEQ ID NO: 1. In some embodiments, the condensate shifting domain comprises at least one modification comprising a truncation of amino acid residues 44-163; truncation of amino acid residues 226-347; truncation of amino acid residues 560-670, W742L mutation, or a combination thereof compared to SEQ ID NO: 1. In some embodiments, the condensate shifting domain comprises at least one modification comprising a truncation of amino acid residues 44-163; truncation of amino acid residues 226-347; truncation of amino acid residues 560-670 and W742L mutation compared to SEQ ID NO: 1. In some embodiments, the condensate shifting domain comprises at least one modification comprising a truncation of amino acid residues 1-293; truncation of amino acid residues 556-666, W742L mutation, or a combination thereof compared to SEQ ID NO: 1. In some embodiments, the condensate shifting domain comprises at least one modification comprising a truncation of amino acid residues 1-293; truncation of amino acid residues 556-666, and W742L mutation compared to SEQ ID NO: 1.

In some embodiments, the condensate shifting domain comprises at least one modification comprising a truncation of amino acid residues 44-163; truncation of amino acid residues 226-347; truncation of amino acid residues 560-670, or a combination thereof compared to SEQ ID NO: 9. In some embodiments, the condensate shifting domain comprises at least one modification comprising a truncation of amino acid residues 44-163; truncation of amino acid residues 226-347; and truncation of amino acid residues 560-670 compared to SEQ ID NO: 9.

In some embodiments, the condensate shifting domain comprising an amino acid sequence that is at least 70%, at least 80%, at least 90%, at least 95%, or at least 99% identical to an AR or fragment thereof can form a first condensate when the condensate shifting domain is contacted with a condensate modulator described here. The first condensate comprises a non-membrane-encapsulated compartment formed by phase separation (including all stages of phase separation) of one or more of the condensate shifting domains described herein. In some embodiments, the first condensate comprises a liquid-liquid condensate separation (LLPS). In some embodiments, the first condensate modulates the molecules in a cell by concentrating the molecules in the first condensate. In some embodiments, the first condensate can be shifted to a second condensate by contacting the engineered polypeptide with at least one additional condensate modulator. In some embodiments, the second condensate can be reversed back to the first condensate by contacting the engineered polypeptide with a third condensate modulator. In some embodiments, the condensate modulator comprises an AR agonist, an AR antagonist, or an AR inverse agonist.

In some embodiments, the engineered polypeptide, when contacted with a condensate modulator described herein, forms the first condensate by contacting with at least one other condensate shifting domain. For example, the engineered polypeptide can comprise an IDR-containing fragment of NTD of AR, where the IDR-containing fragment is tau-1 or tau-5. Tau-1 covers residues from amino acid positions 102 to 371 of AR, with numbering relative to SEQ ID NO: 1. Tau-5 covers residues from amino acid positions 330 to 448 of AR, with numbering reference to SEQ ID NO: 1. In some embodiments, the IDR-containing fragment is contained within a region from amino acid positions 102 to 371, positions 102 to 300, positions 102 to 250, positions 102 to 200, positions 102 to 150, positions 150 to 300, positions 200 to 300, positions 250 to 300, with numbering relative to SEQ ID NO: 1. In some embodiments, the IDR-containing fragment is contained within a region from amino acid positions 330 to 448, positions 330 to 420, positions 330 to 400, positions 330 to 380, positions 330 to 360, positions 350 to 448, positions 380 to 448, positions 400 to 448, or positions 400 to 420, with numbering relative to SEQ ID NO: 1. In some embodiments, the IDR-containing fragment has a length of about or at least 20 residues, 25 residues, 30 residues, 40 residues, or 50 residues. In some embodiments, the condensate shifting domain binds to a residue within an IDR-containing fragment provided herein. In some embodiments, the condensate shifting domain binds to the IDR of the AR, or binds to a region outside of the IDR but can allosterically affect IDR. Binding to IDR can be determined using suitable methods known in the art. In some embodiments, the region outside of the IDR is within the NTD of AR.

In some embodiments, the first condensate is formed when the engineered polypeptide, upon contacting with a condensate modulator described herein, is operatively contacted with at least one other condensate shifting domain. In some embodiments, the first condensate is formed when the engineered polypeptide is contacted with a condensate modulator comprising a nuclear receptor ligand, including a nuclear receptor agonist, a nuclear receptor antagonist, or a nuclear receptor inverse agonist. In some embodiments, the first condensate is formed when the engineered polypeptide is contacted with a condensate modulator comprising an androgen receptor agonist, an androgen receptor antagonist, or an androgen receptor inverse agonist. In some embodiments, the first condensate is formed when the engineered polypeptide is contacted with a condensate modulator comprising an androgen receptor antagonist such as bicalutamide.

In some embodiments, the formation of the first condensate can be inhibited or reversed by contacting the engineered polypeptide with a condensate modulator described herein. In some embodiments, the first condensate can be inhibited or reversed by contacting the engineered polypeptide with a condensate modulator comprising a nuclear receptor ligand such as a nuclear receptor agonist, a nuclear receptor antagonist, or a nuclear receptor inverse agonist. In some embodiments, the first condensate can be inhibited or reversed by contacting the engineered polypeptide with a condensate modulator comprising an androgen receptor agonist, an androgen receptor antagonist, or an androgen receptor inverse agonist. In some embodiments, the first condensate can be inhibited or reversed by contacting the engineered polypeptide with an androgen receptor antagonist such as enzalutamide. In some embodiments, the first condensate can be inhibited or reversed by contacting the engineered polypeptide with an androgen receptor agonist such as DHT.

### Estrogen Receptor

In some embodiments, the condensate shifting domain described herein comprises an amino acid sequence of estrogen receptor (ER) or fragment thereof. ER is a member of the steroid and nuclear receptor superfamily and is mainly expressed in estrogen target tissues, such as endometrium, breast cancer cells, ovarian stromal cells, and the hypothalamus. ER is a soluble protein that functions as an intracellular transcriptional factor. Upon binding and activation by estrogens, ER undergoes conformational changes and posttranslational modifications, dimerization, nuclear translocation, and ultimately, binding to the regulatory regions of the DNA of target genes, known as estrogen response elements (ERE).

Wild type ER has two classes: estrogen receptor alpha (ERα) and estrogen receptor beta (ERβ). ER, similar to AR, has a DNA binding domain (DBD) and an estrogen-dependent ligand-binding domain (LBD). The full length wild type ER has an amino acid sequence of SEQ ID NO: 5. The term “estrogen receptor” or “ER” as used herein encompasses both wild type ER or ER variant (e.g., ER comprising at least one modification). Wild type ER as used herein refers to the full length FR, having an exemplary sequence of SEQ ID NO: 5. ER variant as used herein encompasses all different forms of ER that retains at least part of the N-terminus domain and can phase separate or can form condensates. Examples of ER variants include, without limitation, mutants, fragments, fusions, and splicing variants. In some embodiments, the ER variant is resistant to at least one estrogen-deprivation therapy.

In some embodiments, the ER variant comprises one or more mutations. In some embodiments, the ER variant comprises one or more mutations in ligand binding domain (LBD). In some embodiments, the one or more mutations in the LBD results in gain of function in ER. In some embodiments, the ER variant comprises one or more mutations at the residues selected from the group consisting of K210A, R211E, L379R, G521R, C400V, M543A, and L544A, wherein the numbering is relative to SEQ ID NO: 5. In some embodiments, the ER variant comprises truncation of amino acid residues 250-274 or L379R mutation compared to SEQ ID NO: 5. In some embodiments, the ER variant comprises truncation of amino acid residues 250)-274 and L379R mutation compared to SEQ ID NO: 5. In some embodiments, the ER variant comprises truncation of amino acid residues 250-274 or G521R mutation compared to SEQ ID NO: 5. In some embodiments, the ER variant comprises truncation of amino acid residues 250-274 and G521R mutation compared to SEQ ID NO: 5. In some embodiments, the ER variant comprises truncation of amino acid residues 250-274; C400V mutation; M543A mutation; L544A mutation; or a combination thereof compared to SEQ ID NO: 5. In some embodiments, the ER variant comprises truncation of amino acid residues 250-274; C400V mutation; M543A mutation; and L544A mutation compared to SEQ ID NO: 5. In some embodiments, the ER variant comprises K210A mutation; R211E mutation; L379R mutation; G521R mutation; C400V mutation; M543A mutation; L544A mutation; truncation of amino acid residues 250-274; or a combination thereof compared to SEQ ID NO: 5.

In some embodiments, the ER variant lacks all or part of DBD. For example, the ER variant lacks DBD encoded by SEQ ID NO: 7. In some embodiments, the ER variant lacks all or part of DBD comprises an amino acid sequence that is at least 70%, at least 80%, at least 90%, at least 95%, or at least 99% identical to SEQ ID NO: 8. In some embodiments, the engineered polypeptide comprises at least one modification comprising K210A, R211E, and G521R mutation compared to SEQ ID NO: 5. In some embodiments, the engineered polypeptide comprises the at least one modification comprising K210A, R211E. L379R, C400V, M543A, and L544A mutation compared to SEQ ID NO: 5. In some embodiments, the engineered polypeptide comprises the at least one modification comprising truncation of amino acid residues 250-274 and L379R mutation compared to SEQ ID NO: 5. In some embodiments, the engineered polypeptide comprises the at least one modification comprising truncation of amino acid residues 250-274 and G521R mutation compared to SEQ ID NO: 5. In some embodiments, the engineered polypeptide comprises the at least one modification comprising truncation of amino acid residues 250-274 and C400V, M543A, and L544A mutation compared to SEQ ID NO: 5. In some embodiments, the engineered polypeptide comprises the at least one modification comprises truncation of amino acid residues 44-163; truncation of amino acid residues 226-347; and truncation of amino acid residues 560-670 compared to SEQ ID NO: 9. In some embodiments, the engineered polypeptide comprises the at least one modification comprising truncation of amino acid residues 250-274 compared to SEQ ID NO: 10.

In some embodiments, the condensate shifting domain comprising an amino acid sequence that is at least 70%, at least 80%, at least 90%, at least 95%, or at least 99% identical to an ER or fragment thereof can form a first condensate when the condensate shifting domain is contacted with a condensate modulator described here. The first condensate comprises a non-membrane-encapsulated compartment formed by phase separation (including all stages of phase separation) of one or more of the condensate shifting domains described herein. In some embodiments, the first condensate comprises a liquid-liquid condensate separation (LLPS). In some embodiments, the first condensate modulates the molecules in a cell by concentrating the molecules in the first condensate. In some embodiments, the first condensate can be shifted to a second condensate by contacting the engineered polypeptide with at least one additional condensate modulator. In some embodiments, the second condensate can be reversed back to the first condensate by contacting the engineered polypeptide with a third condensate modulator. In some embodiments, the condensate modulator comprises nuclear receptor ligand comprising an ER agonist, an ER antagonist, or an ER inverse agonist.

In some embodiments, the first condensate is formed when the engineered polypeptide, upon contacting with a condensate modulator described herein, is operatively contacted with at least one other condensate shifting domain. In some embodiments, the first condensate is formed when the engineered polypeptide is contacted with a condensate modulator comprising a nuclear receptor ligand, including, a nuclear receptor agonist, a nuclear receptor antagonist, or a nuclear receptor inverse agonist. In some embodiments, the first condensate is formed when the engineered polypeptide is contacted with a condensate modulator comprising an estrogen receptor agonist, an estrogen receptor antagonist, or an estrogen receptor inverse agonist. In some embodiments, the first condensate is formed when the engineered polypeptide is contacted with a condensate modulator comprising an estrogen receptor antagonist such as tamoxifen.

In some embodiments, the first condensate can be shifted to a second condensate by contacting the engineered polypeptide with an additional condensate modulator described herein. In some embodiments, the first condensate can be shifted to the second condensate by contacting the engineered polypeptide with a condensate modulator comprising a nuclear receptor ligand such as a nuclear receptor agonist, a nuclear receptor antagonist, or a nuclear receptor inverse agonist. In some embodiments, the first condensate can be shifted to the second condensate by contacting the engineered polypeptide with a condensate modulator comprising an estrogen receptor agonist, an estrogen receptor antagonist, or an estrogen receptor inverse agonist. In some embodiments, the first condensate can be shifted to the second condensate by contacting the engineered polypeptide with an estrogen receptor antagonist such as fulvestrant.

### Condensate Modulator

Described herein, in some aspects, are condensate modulators. In some embodiments, the condensate modulator induces the formation of a condensate describe herein. For example, the condensate modulator, when contacted with an engineered polypeptide described herein, can induce formation of a first condensate or a second condensate by operatively contacted the engineered polypeptide and at least one other condensate shifting domain. In some embodiments, the condensate modulator reverses a condensate described herein. For example, a first condensate or a second condensate can be reversed by the condensate modulator contacting with an engineered polypeptide, where the engineered polypeptide is part of the first condensate or the second condensate. In some embodiments, one or more condensate modulators can be contacted with the same engineered polypeptide to induce formation or shifting of the condensates. For example, a first condensate modulator can be in contact with an engineered polypeptide, thereby operatively contacting the engineered polypeptide with at least one other condensate shifting domain to form a first condensate. Additionally, an additional condensate modulator can be in contact with the engineered polypeptide to either reverse the first condensate or shift the first condensate to a second condensate. FIG. 2 illustrates examples of the shifting between the different condensates in a cell meditated by the condensate shifting domain and a condensate modulator described herein. In some embodiment the condensate modulator can be the second condensate modulator, the third condensate modulator, or the fourth condensate modulator described herein.

In some embodiments, the condensate modulator comprises a molecule such as a compound, a small molecule, a peptide, a nucleic acid, a sugar, a lipid, or a combination thereof. In some embodiments, the condensate modulator is a ligand that is endogenous to a cell. In some embodiments, the condensate modulator is a ligand that is exogenous to a cell. In some embodiments, the condensate modulator comprises a functional domain ligand. In some embodiments, the functional domain ligand can be a functional domain agonist. In some embodiments, the functional domain ligand can be a functional domain antagonist. In some embodiments, the functional domain ligand can be a functional domain inverse agonist. In some embodiments, the functional domain ligand can be an antigen for binding to a functional domain comprising CAR or TCR. Other exemplary functional domain ligand can include receptor tyrosine kinase ligand such as insulin growth factor (IGF), epidermal growth factor (EGF), platelet-derived growth factor (PDGF), or fibroblast growth factor (FGF).

In some embodiments, the condensate modulator comprises a nuclear receptor ligand. In some embodiments, the nuclear receptor ligand is an endogenous ligand to a cell. In some embodiments, the nuclear receptor ligand is an exogenous ligand to a cell. In some embodiments, the nuclear receptor ligand comprises a nuclear receptor agonist, a nuclear receptor antagonist, or a nuclear receptor inverse agonist. For example, the nuclear receptor ligand can bind to and activate nuclear receptors include lipophilic substances such as endogenous hormones, vitamins A and D, and xenobiotic hormones. In some instances, the nuclear receptor ligand can be synthetic. For example, nuclear receptor ligand can be synthetic compound for binding to a condensate shifting domain comprising a nuclear receptor or fragment thereof. In some embodiments, the synthetic compound can bind to a condensate shifting domain comprising an orphan or adopted orphan nuclear receptor or fragment thereof. Other nuclear receptor ligand for binding to a condensate shifting domain comprising an orphan receptor such as FXR (farnesoid X receptor), LXR (liver X receptor), and PPAR (peroxisome proliferator-activated receptor) can include a number of metabolic intermediates such as fatty acids, bile acids, or sterols with relatively low affinity.

In some embodiments, the condensate modulator comprises an androgen receptor agonist. In some embodiments, the condensate modulator comprises an androgen receptor antagonist. In some embodiments, the condensate modulator comprises an androgen receptor inverse agonist. In some embodiments, the condensate modulator comprises endogenous androgens (e.g., testosterone, dihydrotestosterone, androstenedione, androstenediol, or dehydroepiandrosterone); synthetic androgens (e.g., methyltestosterone, metandienone, nandrolone, trenbolone, oxandrolone, orstanozolol); selective androgen receptor modulators (e.g., andarine or enobosarm); steroidal antiandrogens (e.g., cyproterone acetate, chlormadinone acetate, spironolactone, or oxendolone); nonsteroidal antiandrogens (e.g., flutamide, nilutamide, bicalutamide, enzalutamide, apalutamide, or RU-58841); or N-terminal domain antiandrogens (e.g., bisphenol A, EPI-001, ralaniten, or JN compounds). In some embodiments, the condensate modulator is bicalutamide. In some embodiments, bicalutamide, when contacted with an engineered polypeptide described herein (e.g., an engineered polypeptide comprising a condensate shifting domain comprising an AR or fragment thereof), operatively contacts the engineered polypeptide with at least one other condensate shifting domain to form a first condensate. In some embodiments, the condensate modulator is enzalutamide. In some embodiments, enzalutamide, when contacted with the engineered polypeptide, reverses the formation of the first condensate formed between the operatively contacting of the engineered polypeptide and the at least one other condensate shifting domain.

In some embodiments, the condensate modulator comprises an estrogen receptor agonist. In some embodiments, the condensate modulator comprises an estrogen receptor antagonist. In some embodiments, the condensate modulator comprises an estrogen receptor inverse agonist. In some embodiments, the condensate modulator comprises endogenous estrogen (e.g., estradiol, estrone, estriol, or estetrol); natural estrogen (e.g., conjugated estrogen); synthetic estrogen (e.g., ethinylestradiol or diethylstilbestrol): mixed estrogen (agonist and antagonist mode of action); phytoestrogens (e.g., coumestrol, daidzein, genistein, or miroestrol); selective estrogen receptor modulators (e.g., tamoxifen, clomifene, or raloxifene); antiestrogens (e.g., fulvestrant, ICI-164384, or ethamoxytriphetol). In some embodiments, the condensate modulator is tamoxifen. In some embodiments, tamoxifen, when contacted with an engineered polypeptide described herein (e.g., an engineered polypeptide comprising a condensate shifting domain comprising ER or fragment thereof, operatively contacts the engineered polypeptide with at least one other condensate shifting domain to form a first condensate. In some embodiments, the condensate modulator is fulvestrant. In some embodiments, fulvestrant, when contacted with the engineered polypeptide, shifts the first condensate formed between the operatively contacting of the engineered polypeptide and the at least one other condensate shifting domain to a second condensate, where the first condensate and the second condensate are different condensates. In some embodiments, the first condensate allows exchange of molecules between inside and outside of the first condensate. In some embodiments, the second condensate reduce exchange of molecules between inside and outside of the second condensate. As such, the shift from the first condensate to the second condensate can decrease or inhibit enzymatic activity associated with the molecules modulated by the first condensate or the second condensate.

### Method of Modulating Molecule

Described herein, in some aspects, are methods of delivering an engineered polypeptide, a system comprising the engineered polypeptide, or an engineered polynucleotide encoding the engineered polypeptide described herein to a cell. In some aspects, the method comprises delivering directly or indirectly the engineered polypeptide, the system, or the engineered polynucleotide to the cell. In some aspects, the method comprises delivering the cell with an engineered polynucleotide, where the cell can then express the engineered polypeptide described herein. In some aspects, the engineered polynucleotide can be delivered into the cell via any of the transfection methods described herein. In some aspects, the engineered polynucleotide can be delivered into the cell via the use of expression vectors such as viral vectors. In the context of an expression vector, the vector can be readily introduced into the cell described herein by any method in the art. For example, the expression vector can be transferred into the cell by physical, chemical, or biological means.

Physical methods for introducing the engineered polynucleotide or vector encoding the engineered polynucleotide into the cell can include calcium phosphate precipitation, lipofection, particle bombardment, microinjection, gene gun, electroporation, and the like. Methods for producing cells comprising vectors and/or exogenous nucleic acids are suitable for methods herein. One method for the introduction of engineered polynucleotide or vector encoding the engineered polynucleotide into a host cell is calcium phosphate transfection.

Chemical means for introducing the engineered polynucleotide or vector encoding the engineered polynucleotide into the cell can include colloidal dispersion systems, such as macromolecule complexes, nanocapsules, microspheres, beads, and lipid-based systems including oil-in-water emulsions, micelles, mixed micelles, spherical nucleic acid (SNA), liposomes, or lipid nanoparticles. An exemplary colloidal system for use as a delivery vehicle in vitro and in vivo is a liposome (e.g., an artificial membrane vesicle). Other methods of state-of-the-art targeted delivery of nucleic acids are available, such as delivery of engineered polynucleotide or vector encoding the engineered polynucleotide with targeted nanoparticles or other suitable sub-micron sized delivery system.

In the case where a non-viral delivery system is utilized, an exemplary delivery vehicle is a liposome. The use of lipid formulations is contemplated for the introduction of the oligonucleotide or vector encoding the oligonucleotide into a cell (in vitro, ex vivo or in vivo). In another aspect, the engineered polynucleotide or vector encoding the engineered polynucleotide can be associated with a lipid. The engineered polynucleotide or vector encoding the engineered polynucleotide associated with a lipid, in some aspects, is encapsulated in the aqueous interior of a liposome, interspersed within the lipid bilayer of a liposome, attached to a liposome via a linking molecule that is associated with both the liposome and the oligonucleotide, entrapped in a liposome, complexed with a liposome, dispersed in a solution containing a lipid, mixed with a lipid, combined with a lipid, contained as a suspension in a lipid, contained or complexed with a micelle, or otherwise associated with a lipid. Lipid, lipid/DNA or lipid/expression vector associated compositions are not limited to any particular structure in solution. For example, In some aspects, they are present in a bilayer structure, as micelles, or with a “collapsed” structure. Alternately, they are simply interspersed in a solution, possibly forming aggregates that are not uniform in size or shape. Lipids are fatty substances which are, in some aspects, naturally occurring or synthetic lipids. For example, lipids include the fatty droplets that naturally occur in the cytoplasm as well as the class of compounds which contain long-chain aliphatic hydrocarbons and their derivatives, such as fatty acids, alcohols, amines, amino alcohols, and aldehydes. Lipids suitable for use are obtained from commercial sources. Stock solutions of lipids in chloroform or chloroform/methanol are often stored at about −20° C. Chloroform is used as the only solvent since it is more readily evaporated than methanol. “Liposome” is a generic term encompassing a variety of single and multilamellar lipid vehicles formed by the generation of enclosed lipid bilayers or aggregates. Liposomes are often characterized as having vesicular structures with a phospholipid bilayer membrane and an inner aqueous medium. Multilamellar liposomes have multiple lipid layers separated by aqueous medium. They form spontaneously when phospholipids are suspended in an excess of aqueous solution. The lipid components undergo self-rearrangement before the formation of closed structures and entrap water and dissolved solutes between the lipid bilayers. However, compositions that have different structures in solution than the normal vesicular structure are also encompassed. For example, the lipids, In some aspects, assume a micellar structure or merely exist as nonuniform aggregates of lipid molecules. Also contemplated are lipofectamine-nucleic acid complexes.

In some cases, non-viral delivery method comprises lipofection, nucleofection, microinjection, biolistics, virosomes, liposomes, immunoliposomes, exosomes, polycation or lipid: cargo conjugates (or aggregates), naked polypeptide (e.g., recombinant polypeptides), naked DNA, artificial virions, and agent-enhanced uptake of polypeptide or DNA. In some aspects, the delivery method comprises conjugating or encapsulating the compositions or the oligonucleotides described herein with at least one polymer such as natural polymer or synthetic materials. The polymer can be biocompatible or biodegradable. Non-limiting examples of suitable biocompatible, biodegradable synthetic polymers can include aliphatic polyesters, poly(amino acids), copoly(ether-esters), polyalkylenes oxalates, polyamides, poly(iminocarbonates), polyorthoesters, polyoxaesters, polyamidoesters, polyoxaesters containing amine groups, and poly(anhydrides). Such synthetic polymers can be homopolymers or copolymers (e.g., random, block, segmented, graft) of a plurality of different monomers, e.g., two or more of lactic acid, lactide, glycolic acid, glycolide, epsilon-caprolactone, trimethylene carbonate, p-dioxanone, etc. In an example, the scaffold can be comprised of a polymer comprising glycolic acid and lactic acid, such as those with a ratio of glycolic acid to lactic acid of 90/10 or 5/95. Non-limiting examples of naturally occurring biocompatible, biodegradable polymers can include glycoproteins, proteoglycans, polysaccharides, glycosamineoglycan (GAG) and fragment(s) derived from these components, elastin, laminins, decrorin, fibrinogen/fibrin, fibronectins, osteopontin, tenascins, hyaluronic acid, collagen, chondroitin sulfate, heparin, heparan sulfate, ORC, carboxymethyl cellulose, and chitin.

In aspects, the engineered polynucleotide described herein can be delivered into a cell as a vector such as a viral vector. Viral vectors, and especially retroviral vectors, have become the most widely used method for inserting genes into mammalian, e.g., human cells. Other viral vectors, in some embodiments, are derived from lentivirus, poxviruses, herpes simplex virus I, adenoviruses and adeno-associated viruses, and the like. Exemplary viral vectors include retroviral vectors, adenoviral vectors, adeno-associated viral vectors (AAVs), pox vectors, parvoviral vectors, baculovirus vectors, measles viral vectors, or herpes simplex virus vectors (HSVs). In some instances, the retroviral vectors include gamma-retroviral vectors such as vectors derived from the Moloney Murine Leukemia Virus (MoMLV, MMLV, MuLV, or MLV) or the Murine Stem cell Virus (MSCV) genome. In some instances, the retroviral vectors also include lentiviral vectors such as those derived from the human immunodeficiency virus (HIV) genome. In some instances, AAV vectors include AAV1, AAV2, AAV4, AAV5, AAV6, AAV7, AAV8, or AAV9 serotype. In some instances, viral vector is a chimeric viral vector, comprising viral portions from two or more viruses. In additional instances, the viral vector is a recombinant viral vector.

In some cases, the engineered polynucleotide encoding the engineered polypeptide described herein can be administered to the subject in need thereof via the use of the transgenic cells generated by introduction of the engineered polynucleotide first into allogeneic or autologous cells. In some cases, the cell can be isolated. In some aspects, the cell can be isolated from the subject.

In some embodiments, the engineered polypeptide, upon contact with a condensate modulator, can form a first condensate or a second condensate by the engineered polypeptide operatively contacted with at least one other condensate shifting domain. The formation of the first condensate or the second condensate can be determined based suitable methods such as Transfer Diffusion Nuclear Magnetic Resonance (STD NMR), affinity assay, mass spectrometry analysis, pull down assay, or live cell imaging via deconvolution microscopy, structured illumination microscopy, turbidity detection, optical tweezer systems, microfluidic systems, or interference microscopy.

In some embodiments, the formation of the first condensate or the second condensate can be determined by live cell imaging. For example, the engineered polypeptide can be conjugated with a detectable label such as a fluorescent molecule, which allows the engineered polypeptide to be visualized under microscope, and the fluorescence intensity can indicate formation of the first condensate or the second condensate. For example, in absence of the formation of the first condensate or the second condensate, the fluorescence intensity of the labeled engineered polypeptide generally appear evenly distributed in nucleus or cytosol. However, upon the engineered polypeptide contacted with the condensate modulator, formation of the first condensate or the second condensate can be observed as bright spots or puncta in contrast to background, where the bright spots or puncta indicates presence of the first condensate or the second condensate.

In some embodiments, the first condensate or the second condensate forms primarily in a nucleus of a cell. In some embodiments, the first condensate or the second condensate forms in a nucleus of a cell. In some embodiments, the first condensate or the second condensate forms primarily in an organelle of a cell. In some embodiments, the first condensate or the second condensate forms in an organelle of a cell. In some embodiments, the first condensate or the second condensate forms primarily in cytoplasm of a cell. In some embodiments, the first condensate or the second condensate forms in cytoplasm of a cell. In some embodiments, the first condensate or the second condensate forms primarily in cytoplasmic membrane of a cell. In some embodiments, the first condensate or the second condensate forms in cytoplasmic membrane of a cell. In some embodiments, the first condensate or the second condensate forms in cytoplasm due to the engineered polypeptide having the at least one modification. For example, the engineered polypeptide comprising the condensate shifting domain comprising the AR variant or the ER variant forms the first condensate or the second condensate in the cytoplasm as opposed to the condensate formed by wild type AR or ER, which occur in the nucleus of the cell.

In some embodiments, the first condensate can be used for sensing fast, adaptive, or reversible responses. In some embodiments, the first condensate can be used to buffer concentrations of molecules such as the molecules being modulated by the first condensate. Increasing the concentration of the modulated molecules results in condensation of the modulated molecules above a saturation concentration. In some embodiments, the first condensate can be used to locally concentrate molecules in various compartments or locations in a cell. Increasing the local concentration of a key enzyme or protein complex can accelerate biochemical reactions. In some embodiments, the first condensate can be used prevent reactions or for inactivation. If one critical component is recruited into the first condensate, but all other components for an enzymatic reaction or signaling event remain outside the first condensate, the reaction or signaling event will be inhibited or slowed down. In some embodiments, the first condensate can be used to form physico-chemical and mechanical filters where pore sizes are determined by the number and dynamics of the cross-links between the pores of the first condensate.

In some embodiments, the first condensate comprises a liquid condensate. In some embodiments, the liquid condensate comprises liquid-liquid condensate separation (LLPS). In some embodiments, the liquid condensate comprises a condensate. In some embodiments, the liquid condensate comprises a membraneless compartment. In some embodiments, the first condensate comprising the liquid condensate can coalesce with other liquid condensate or droplet in the cell. In some embodiments, the first condensate comprising the liquid condensate does not have a well-defined shape. In some embodiments, the first condensate comprising the liquid condensate comprises a spheroid shape. In some embodiments, the first condensate comprising the liquid condensate comprises an amorphous shape. In some embodiments, the first condensate comprises a fluidity as determined by turbidity, viscosity, surface tension, or a combination thereof.

In some embodiments, the first condensate can modulate molecules in the cell by accumulating molecules in the first condensate, thereby increasing concentration of the molecules in the cell. In some embodiments, the increased concentration of the modulated molecules in the first condensate can increase enzymatic activity associated with the modulated molecules. In some embodiments, the first condensate allows movement of the molecules inside the first condensate. In some embodiments, the first condensate allows exchange of the molecules between inside and outside of the first condensate. Molecules modulated by the first condensate (or the second condensate) can be exogenous molecules, endogenous molecules, or a combination thereof. In some embodiments, the modulated molecules comprise at least one enzyme, at least one compound, at least one biomolecule, at least one nucleic acid, at least one lipid, at least one protein, at least one sugar, at least one metabolite, or a combination thereof. In some embodiments, the modulated molecules comprise at least at least one transcription factor.

In some embodiments, the first condensate increases an enzymatic activity by modulating and brining two or more of the molecules to close proximity in the first condensate, thereby increasing enzymatic activity associated with the modulated molecules. In some embodiments, the first condensate modulates concentration of at least one molecule in a cell by concentrating or sequestering the at least molecule in the first condensate. In some embodiments, the concentration of the at least one molecule inside the first condensate is increased relatively to the concentration of the at least one molecule outside the first condensate. In some embodiments, the concentration is increased by at least 70%, at least 80%, at least 90%, at least 95%, or at least 99%. In some embodiments, the concentration of the at least one molecule inside the first condensate is decreased relatively to the concentration of the at least one molecule outside the first condensate. In some embodiments, the concentration is decreased by at least 70%, at least 80%, at least 90%, at least 95%, or at least 99%. In some embodiments, the first condensate is at least 10% more viscous, at least 20% more viscous, at least 30% more viscous, at least 40% more viscous, at least 50% more viscous, at least 60% more viscous, at least 70% more viscous, at least 80% more viscous, at least 90% more viscous, or at least 99% more viscous compared to a comparable first condensate formed by a comparable polypeptide without the at least one modification. In some embodiments, the first condensate is at least 10% less viscous, at least 20% less viscous, at least 30% less viscous, at least 40% less viscous, at least 50% less viscous, at least 60% less viscous, at least 70% less viscous, at least 80% less viscous, at least 90% less viscous, or at least 99% less viscous compared to a comparable first condensate formed by a comparable polypeptide without the at least one modification. In some embodiments, the first condensate comprises at least 10% more surface tension, at least 20% more surface tension, at least 30% more surface tension, at least 40% more surface tension, at least 50% more surface tension, at least 60% more surface tension, at least 70% more surface tension, at least 80% more surface tension, at least 90% more surface tension, or at least 99% more surface tension compared to a comparable first condensate formed by a comparable polypeptide without the at least one modification. In some embodiments, the first condensate comprises at least 10% less surface tension, at least 20% less surface tension, at least 30% less surface tension, at least 40% less surface tension, at least 50% less surface tension, at least 60% less surface tension, at least 70% less surface tension, at least 80% less surface tension, at least 90% less surface tension, or at least 99% less surface tension compared to a comparable first condensate formed by a comparable polypeptide without the at least one modification. In some embodiments, the first condensate comprises at least 10% more turbidity, at least 20% more turbidity, at least 30% more turbidity, at least 40% more turbidity, at least 50% more turbidity, at least 60% more turbidity, at least 70% more turbidity, at least 80% more turbidity, at least 90% more turbidity, or at least 99% more turbidity compared to a comparable first condensate formed by a comparable polypeptide without the at least one modification. In some embodiments, the first condensate comprises at least 10% less turbidity, at least 20% less turbidity, at least 30% less turbidity, at least 40% less turbidity, at least 50% less turbidity, at least 60% less turbidity, at least 70% less turbidity, at least 80% less turbidity, at least 90% less turbidity, or at least 99% less turbidity compared to a comparable first condensate formed by a comparable polypeptide without the at least one modification. In some embodiments, the first condensate increases a rate of exchange of molecule between inside and outside of the first condensate by at least 10%, at least 20%, at least 30%, at least 40%, at least 50%, at least 60%, at least 70%, at least 80%, at least 90%, or at least 99% compared to a rate of exchange of molecule between inside and outside of a comparable first condensate formed by a comparable polypeptide without the at least one modification. In some embodiments, the first condensate decreases a rate of exchange of molecule between inside and outside of the first condensate by at least 10%, at least 20%, at least 30%, at least 40%, at least 50%, at least 60%, at least 70%, at least 80%, at least 90%, or at least 99% compared to a rate of exchange of molecule between inside and outside of a comparable first condensate formed by a comparable polypeptide without the at least one modification. In some embodiments, the first condensate is at least 10%, at least 20%, at least 30%, at least 40%, at least 50%, at least 60%, at least 70%, at least 80%, at least 90%, or at least 99% more likely to accumulate in cytoplasm of a cell compared to a comparable first condensate formed by a comparable polypeptide without the at least one modification.

In some embodiments, the first condensate can be shifted to a second condensate described herein. For example, the first can be shifted to the second condensate by contacting the engineered polypeptide comprising a condensate shifting domain comprising an ER or fragment thereof with a condensate modulator comprising fulvestrant. In some embodiments, the second condensate comprises a condensate that reduce movement of molecules inside the second condensate. In some embodiments, the second condensate comprises a condensate that reduce exchange of molecules between inside and outside of the second condensate. In some embodiments, the second condensate comprises a solid condensate. In some embodiments, the second condensate comprises a semi-solid condensate. In some embodiments, the second condensate comprises a gel condensate. In embodiments, the second condensate can be reversed back to the first condensate by contacting the engineered polypeptide with at least one additional condensate modulator. In some embodiments, the second condensate can be dissolved by contacting the engineered polypeptide with at least one additional condensate modulator

### Method of Treatment

Provided herein are methods for treating a disease or condition in a subject by administering an engineered polypeptide, an engineered polynucleotide, a cell transduced with the engineered polynucleotide, a system, a pharmaceutical composition, or a combination thereof to the subject. In some embodiments, administration is by any suitable mode of administration, including systemic administration (e.g., intravenous, inhalation, etc.). In some embodiments, the subject is human. In some embodiments, the disease or condition comprises: CAR related disease such as hematological malignancies, malignancies, leukemia, multiple myeloma, malignant lymphoma, and so on): kinase related disease such as autoimmune diseases, heteroimmune diseases, cancers, or thromboembolic disease TCR related disease such as hematological malignancies, malignancies, leukemia, pancreatic malignancy, malignant lymphoma, and so on: metabolic disease such as protein metabolism disorders, glucose metabolism disorder, diabetes, ester metabolism disorder, water and electrolyte metabolism disorders, metabolic disorders of inorganic elements, purine metabolism disorders, diabetic ketoacidosis, Hyperglycemia Hyperosmolar Syndrome, Hypoglycemia, Gout, Protein-Energy. Malnutrition, Vitamin A deficiency, Scurvy, Vitamin D deficiency, or steoporosis; transcription factor related disease such as breast cancer, prostate cancers, uterine myoma, lung cancer, Head and neck cancer, leukemia, pancreatic malignancy, hematological malignancies, malignancies, lymphoma, and so on.

In some embodiments, the engineered polypeptide, the engineered polynucleotide, the cell transduced with the engineered polynucleotide, the system, the pharmaceutical composition, or the combination thereof may be administered to a subject in a suitable dose, mode of administration, and frequency, depending on the intended effect. In some embodiments, such administration is performed at least once during a period of time (e.g., every 2 days, twice a week, once a week, every week, three times per month, two times per month, one time per month, every 2 months, every 3 months, every 4 months, every 5 months, every 6 months, every 7 months, every 8 months, every 9 months, every 10 months, every 11 months, once a year). In some embodiments, the composition is administered two or more times (e.g., 2, 3, 4, 5, 6, 7, 8, 9, 10, 11, 12, 13, 14, 15, 16, 17, 18, 19, 20, 30, 40, 50, 60, 70, 80, 90, 100 times) during a period of time. In some embodiments, such administration comprises administering in a therapeutically-effective amount by various forms and routes including, for example, oral, or topical administration. In some embodiments, the engineered polypeptide, the engineered polynucleotide, the cell transduced with the engineered polynucleotide, the system, the pharmaceutical composition, or the combination thereof may be administered by parenteral, intravenous, subcutaneous, intramuscular, intradermal, intraperitoneal, intracerebral, subarachnoid, intraocular, intrasternal, ophthalmic, endothelial, local, intranasal, intrapulmonary, rectal, intraarterial, intrathecal, inhalation, intralesional, intradermal, epidural, intracapsular, subcapsular, intracardiac, transtrachcal, subcuticular, subarachnoid, or intraspinal administration, e.g., injection or infusion. In some embodiments, a composition may be administered by absorption through epithelial or mucocutaneous linings (e.g., oral mucosa, rectal and intestinal mucosa administration). In some embodiments, the engineered polypeptide, the engineered polynucleotide, the cell transduced with the engineered polynucleotide, the system, the pharmaceutical composition, or the combination thereof is delivered via multiple administration routes.

The engineered polypeptide, the engineered polynucleotide, the cell transduced with the engineered polynucleotide, the system, the pharmaceutical composition, or the combination thereof may be administered in a local manner, for example, via injection of the agent directly into an organ, optionally in a depot or sustained release formulation or implant. The engineered polypeptide, the engineered polynucleotide, the cell transduced with the engineered polynucleotide, the system, the pharmaceutical composition, or the combination thereof may be provided in the form of a rapid release formulation, in the form of an extended release formulation, or in the form of an intermediate release formulation.

The engineered polypeptide, the engineered polynucleotide, the cell transduced with the engineered polynucleotide, the system, the pharmaceutical composition, or the combination thereof provided herein may be administered in conjunction with other therapies such as an antiviral therapy, a chemotherapy, an antibiotic, a cell therapy, a cytokine therapy, or an anti-inflammatory agent. Actual dosage levels may be varied so as to obtain an amount of the agent to achieve the desired therapeutic response for a particular subject, composition, and mode of administration, without being toxic to the subject. Dosage regimens may be adjusted to provide the optimum desired response (e.g., a therapeutic and/or prophylactic response). For example, a single bolus may be administered, several divided doses may be administered over time or the dose may be proportionally reduced or increased as indicated by the exigencies of the therapeutic situation. It is especially advantageous to formulate parenteral compositions in dosage unit form for case of administration and uniformity of dosage. Dosage unit form as used herein refers to physically discrete units suited as unitary dosages for the subjects (e.g., the subjects for immunization or the subjects for treatment); each unit contains a predetermined quantity of active agent calculated to produce the desired therapeutic effect in association with the required pharmaceutical carrier. In some embodiments, the engineered polypeptide, the engineered polynucleotide, the cell transduced with the engineered polynucleotide, the system, the pharmaceutical composition, or the combination thereof can be repeatedly administered to the subject in need thereof.

In some cases, an administration of the engineered polypeptide, the engineered polynucleotide, the cell transduced with the engineered polynucleotide, the system, the pharmaceutical composition, or the combination thereof is sufficient to decrease at least a symptom of a disease or condition, treat the disease or condition, and/or eliminate the disease or condition. In some cases, improvements of diseases or conditions can be ascertained by any of the provided diagnostic assays.

### Pharmaceutical Composition

Described herein are pharmaceutical compositions comprising an engineered polypeptide, an engineered polynucleotide, a cell transduced with the engineered polypeptide, a system, or a combination thereof. In some embodiments, the pharmaceutical compositions further comprise a pharmaceutically acceptable: carrier, excipient, diluent, or nebulized inhalant. In some embodiments, the pharmaceutical composition comprises at least one condensate modulator. In some embodiments, the pharmaceutical composition comprises at least one additional active agent. In some embodiments, the active agents may be a chemotherapeutic agent, a cytotoxic agent, a cytokine, a growth-inhibitory agent, an anti-hormonal agent, an anti-angiogenic agent, a cardio protectant, or a checkpoint inhibitor.

In practicing the methods of treatment or use provided herein, therapeutically effective amounts of pharmaceutical composition described herein is administered to a subject having a disease, disorder, or condition to be treated, e.g., cancer. In some embodiments, the subject is a human. A therapeutically effective amount may vary widely depending on the severity of the disease, the age and relative health of the subject, the potency of the pharmaceutical composition used and other factors. The pharmaceutical composition described herein, in some aspects, may be used singly or in combination with one or more therapeutic agents as components of mixtures.

The pharmaceutical compositions described herein may be administered to a subject by appropriate administration routes, including but not limited to, intravenous, intraarterial, oral, parenteral, buccal, topical, transdermal, rectal, intramuscular, subcutaneous, intraosseous, transmucosal, inhalation, or intraperitoneal administration routes. The composition described herein may include, but not limited to, aqueous liquid dispersions, self-emulsifying dispersions, solid solutions, liposomal dispersions, aerosols, solid dosage forms, powders, immediate release formulations, controlled release formulations, fast melt formulations, tablets, capsules, pills, delayed release formulations, extended release formulations, pulsatile release formulations, multiparticulate formulations, and mixed immediate and controlled release formulations.

The pharmaceutical composition may be manufactured in a conventional manner such as, by way of example only, by means of conventional mixing, dissolving, granulating, dragee-making, levigating, emulsifying, encapsulating, entrapping or compression processes. The pharmaceutical composition may include at least an exogenous therapeutic agent as an active ingredient in free-acid or free-base form, or in a pharmaceutically acceptable salt form. In addition, the methods and compositions described herein include the use of N-oxides (if appropriate), crystalline forms, amorphous phases, as well as active metabolites of these compounds having the same type of activity. In some embodiments, therapeutic agents exist in unsolvated form or in solvated forms with pharmaceutically acceptable solvents such as water, ethanol, and the like. The solvated forms of the therapeutic agents are also considered to be disclosed herein.

In certain embodiments, pharmaceutical composition provided herein includes one or more preservatives to inhibit microbial activity. Suitable preservatives include mercury-containing substances such as merfen and thiomersal; stabilized chlorine dioxide; and quaternary ammonium compounds such as benzalkonium chloride, cetyltrimethylammonium bromide and cetylpyridinium chloride. In some embodiments, pharmaceutical composition described herein benefits from antioxidants, metal chelating agents, thiol containing compounds and other general stabilizing agents.

The pharmaceutical composition described herein can be formulated into any suitable dosage form, including but not limited to, aqueous oral dispersions, liquids, gels, syrups, elixirs, slurries, suspensions, solid oral dosage forms, aerosols, controlled release formulations, fast melt formulations, effervescent formulations, lyophilized formulations, tablets, powders, pills, dragees, capsules, delayed release formulations, extended release formulations, pulsatile release formulations, multiparticulate formulations, and mixed immediate release and controlled release formulations. In one aspect, a therapeutic agent as discussed herein, e.g., therapeutic agent is formulated into a pharmaceutical composition suitable for intramuscular, subcutaneous, or intravenous injection. In one aspect, formulations suitable for intramuscular, subcutaneous, or intravenous injection include physiologically acceptable sterile aqueous or non-aqueous solutions, dispersions, suspensions or emulsions, and sterile powders for rehydration into sterile injectable solutions or dispersions. Examples of suitable aqueous and non-aqueous carriers, diluents, solvents, or vehicles include water, ethanol, polyols (propyleneglycol, polyethylene-glycol, glycerol, cremophor and the like), suitable mixtures thereof, vegetable oils (such as olive oil) and injectable organic esters such as ethyl oleate. Proper fluidity may be maintained, for example, by the use of a coating such as lecithin, by the maintenance of the required particle size in the case of dispersions, and by the use of surfactants. In some embodiments, formulations suitable for subcutaneous injection also contain additives such as preserving, wetting, emulsifying, and dispensing agents. Prevention of the growth of microorganisms may be ensured by various antibacterial and antifungal agents such as parabens, chlorobutanol, phenol, sorbic acid, and the like. In some cases, it is desirable to include isotonic agents such as sugars, sodium chloride, and the like. Prolonged absorption of the injectable pharmaceutical form may be brought about by the use of agents delaying absorption such as aluminum monostearate and gelatin.

For intravenous injections or drips or infusions, a pharmaceutical composition described herein is formulated in aqueous solutions, preferably in physiologically compatible buffers such as Hank's solution, Ringer's solution, or physiological saline buffer. For transmucosal administration, penetrants appropriate to the barrier to be permeated are used in the formulation. Such penetrants are generally known in the art. For other parenteral injections, appropriate formulations include aqueous or nonaqueous solutions, preferably with physiologically compatible buffers or excipients. Such excipients are known.

Parenteral injections may involve bolus injection or continuous infusion. Pharmaceutical compositions for injection may be presented in unit dosage form, e.g., in ampoules or in multi dose containers, with an added preservative. The composition described herein may be in a form suitable for parenteral injection as a sterile suspensions, solutions or emulsions in oily or aqueous vehicles, and may contain formulatory agents such as suspending, stabilizing and/or dispersing agents. In one aspect, the active ingredient is in powder form for constitution with a suitable vehicle, e.g., sterile pyrogen-free water, before use.

For administration by inhalation, a therapeutic agent is formulated for use as an aerosol, a mist or a powder. Pharmaceutical compositions described herein are conveniently delivered in the form of an aerosol spray presentation from pressurized packs or nebulizers, with the use of a suitable propellant. e.g., dichlorodifluoromethane, trichlorofluoromethane, dichlorotetrafluoroethane, carbon dioxide or other suitable gas. In the case of a pressurized aerosol, the dosage unit may be determined by providing a valve to deliver a metered amount. Capsules and cartridges of such as, by way of example only, gelatin for use in an inhaler or insufflator may be formulated containing a powder mix of the therapeutic agent described herein and a suitable powder base such as lactose or starch. Formulations that include a composition are prepared as solutions in saline, employing benzyl alcohol or other suitable preservatives, fluorocarbons, and/or other solubilizing or dispersing agents known in the art. Preferably these compositions and formulations are prepared with suitable nontoxic pharmaceutically acceptable ingredients. The choice of suitable carriers is dependent upon the exact nature of the nasal dosage form desired, e.g., solutions, suspensions, ointments, or gels. Nasal dosage forms generally contain large amounts of water in addition to the active ingredient. Minor amounts of other ingredients such as pH adjusters, emulsifiers or dispersing agents, preservatives, surfactants, gelling agents, or buffering and other stabilizing and solubilizing agents are optionally present. Preferably, the nasal dosage form should be isotonic with nasal secretions.

Pharmaceutical preparations for oral use are obtained by mixing one or more solid excipient with one or more of the compositions described herein, optionally grinding the resulting mixture, and processing the mixture of granules, after adding suitable auxiliaries, if desired, to obtain tablets or dragee cores. Suitable excipients include, for example, fillers such as sugars, including lactose, sucrose, mannitol, or sorbitol; cellulose preparations such as, for example, maize starch, wheat starch, rice starch, potato starch, gelatin, gum tragacanth, methylcellulose, microcrystalline cellulose, hydroxypropylmethylcellulose, sodium carboxymethylcellulose; or others such as: polyvinylpyrrolidone (PVP or povidone) or calcium phosphate. If desired, disintegrating agents are added such as the cross linked croscarmellose sodium, polyvinylpyrrolidone, agar, or alginic acid or a salt thereof such as sodium alginate. In some embodiments, dyestuffs or pigments are added to the tablets or dragee coatings for identification or to characterize different combinations of active therapeutic agent doses.

In some embodiments, the pharmaceutical compositions of the exogenous therapeutic agents are in the form of a capsules, including push fit capsules made of gelatin, as well as soft, scaled capsules made of gelatin and a plasticizer such as glycerol or sorbitol. The push fit capsules contain the active ingredients in admixture with filler such as lactose, binders such as starches, and/or lubricants such as tale or magnesium stearate and, optionally, stabilizers. In soft capsules, the active therapeutic agent is dissolved or suspended in suitable liquids such as fatty oils, liquid paraffin, or liquid polyethylene glycols. In some embodiments, stabilizers are added. A capsule may be prepared, for example, by placing the bulk blend of the formulation of the therapeutic agent inside of a capsule. In some embodiments, the formulations (non-aqueous suspensions and solutions) are placed in a soft gelatin capsule. In other embodiments, the formulations are placed in standard gelatin capsules or non-gelatin capsules such as capsules comprising HPMC. In other embodiments, the formulation is placed in a sprinkle capsule, wherein the capsule is swallowed whole or the capsule is opened and the contents sprinkled on food prior to eating.

Pharmaceutical compositions for oral administration are in dosages suitable for such administration. In one aspect, solid oral dosage forms are prepared by mixing a composition with one or more of the following: antioxidants, flavoring agents, and carrier materials such as binders, suspending agents, disintegration agents, filling agents, surfactants, solubilizers, stabilizers, lubricants, wetting agents, and diluents. In some embodiments, the solid dosage forms disclosed herein are in the form of a tablet, (including a suspension tablet, a fast-melt tablet, a bite-disintegration tablet, a rapid-disintegration tablet, an effervescent tablet, or a caplet), a pill, a powder, a capsule, solid dispersion, solid solution, bioerodible dosage form, controlled release formulations, pulsatile release dosage forms, multiparticulate dosage forms, beads, pellets, granules. In other embodiments, the composition is in the form of a powder. Compressed tablets are solid dosage forms prepared by compacting the bulk blend of the formulations described above. In various embodiments, tablets will include one or more flavoring agents. In other embodiments, the tablets will include a film surrounding the final compressed tablet. In some embodiments, the film coating may provide a delayed release of a therapeutic agent from the formulation. In other embodiments, the film coating aids in patient compliance. Film coatings typically range from about 1% to about 3% of the tablet weight. In some embodiments, solid dosage forms, e.g., tablets, effervescent tablets, and capsules, are prepared by mixing particles of a therapeutic agent with one or more pharmaceutical excipients to form a bulk blend composition. The bulk blend is readily subdivided into equally effective unit dosage forms such as tablets, pills, and capsules. In some embodiments, the individual unit dosages include film coatings. These formulations are manufactured by conventional formulation techniques.

In another aspect, dosage forms include microencapsulated formulations. In some embodiments, one or more other compatible materials are present in the microencapsulation material. Non-limiting example of materials includes pH modifiers, erosion facilitators, anti-foaming agents, antioxidants, flavoring agents, and carrier materials such as binders, suspending agents, disintegration agents, filling agents, surfactants, solubilizers, stabilizers, lubricants, wetting agents, and diluents.

Liquid formulation dosage forms for oral administration are optionally aqueous suspensions selected from the group including, but not limited to, pharmaceutically acceptable aqueous oral dispersions, emulsions, solutions, elixirs, gels, and syrups. In addition to therapeutic agent the liquid dosage forms optionally include additives such as: (a) disintegrating agents; (b) dispersing agents; (c) wetting agents; (d) at least one preservative, (e) viscosity enhancing agents, (f) at least one sweetening agent, and (g) at least one flavoring agent. In some embodiments, the aqueous dispersions further include a crystal-forming inhibitor.

In some embodiments, the pharmaceutical compositions described herein are self-emulsifying drug delivery systems (SEDDS). Emulsions are dispersions of one immiscible phase in another, usually in the form of droplets. Generally, emulsions are created by vigorous mechanical dispersion. SEDDS, as opposed to emulsions or microemulsions, spontaneously form emulsions when added to an excess of water without any external mechanical dispersion or agitation. An advantage of SEDDS is that only gentle mixing is required to distribute the droplets throughout the solution. Additionally, water or the aqueous condensate is optionally added just prior to administration, which ensures stability of an unstable or hydrophobic active ingredient. Thus, the SEDDS provides an effective delivery system for oral and parenteral delivery of hydrophobic active ingredients. In some embodiments, SEDDS provides improvements in the bioavailability of hydrophobic active ingredients.

Buccal formulations are administered using a variety of formulations known in the art. In addition, the buccal dosage forms described herein may further include a bioerodible (hydrolysable) polymeric carrier that also serves to adhere the dosage form to the buccal mucosa. For buccal or sublingual administration, the compositions may take the form of tablets, lozenges, or gels formulated in a conventional manner.

For intravenous injections, a pharmaceutical composition is optionally formulated in aqueous solutions, preferably in physiologically compatible buffers such as Hank's solution, Ringer's solution, or physiological saline buffer. For transmucosal administration, penetrants appropriate to the barrier to be permeated are used in the formulation. For other parenteral injections, appropriate formulations include aqueous or nonaqueous solutions, preferably with physiologically compatible buffers or excipients.

Parenteral injections optionally involve bolus injection or continuous infusion. Formulations for injection are optionally presented in unit dosage form, e.g., in ampoules or in multi dose containers, with an added preservative. In some embodiments, a composition described herein is in a form suitable for parenteral injection as a sterile suspensions, solutions or emulsions in oily or aqueous vehicles, and contain formulatory agents such as suspending, stabilizing and/or dispersing agents. The compositions for parenteral administration include aqueous solutions of an agent that modulates the activity of a carotid body in water soluble form. Additionally, suspensions of an agent that modulates the activity of a carotid body are optionally prepared as appropriate, e.g., oily injection suspensions.

Conventional formulation techniques include, e.g., one or a combination of methods: (1) dry mixing, (2) direct compression, (3) milling, (4) dry or non-aqueous granulation, (5) wet granulation, or (6) fusion. Other methods include, e.g., spray drying, pan coating, melt granulation, granulation, fluidized bed spray drying or coating (e.g., wurster coating), tangential coating, top spraying, tableting, extruding and the like.

In some embodiments, the compositions are provided that include particles of a therapeutic agent and at least one dispersing agent or suspending agent for oral administration to a subject. The formulations may be a powder and/or granules for suspension, and upon admixture with water, a substantially uniform suspension is obtained.

Furthermore, the pharmaceutical compositions optionally include one or more pH adjusting agents or buffering agents, including acids such as acetic, boric, citric, lactic, phosphoric and hydrochloric acids; bases such as sodium hydroxide, sodium phosphate, sodium borate, sodium citrate, sodium acetate, sodium lactate and tris-hydroxymethylaminomethane; and buffers such as citrate/dextrose, sodium bicarbonate and ammonium chloride. Such acids, bases and buffers are included in an amount required to maintain pH of the composition in an acceptable range.

Additionally, the pharmaceutical compositions optionally include one or more salts in an amount required to bring osmolality of the composition into an acceptable range. Such salts include those having sodium, potassium or ammonium cations and chloride, citrate, ascorbate, borate, phosphate, bicarbonate, sulfate, thiosulfate or bisulfite anions; suitable salts include sodium chloride, potassium chloride, sodium thiosulfate, sodium bisulfite and ammonium sulfate.

Other the pharmaceutical compositions optionally include one or more preservatives to inhibit microbial activity. Suitable preservatives include mercury-containing substances such as merfen and thiomersal; stabilized chlorine dioxide; and quaternary ammonium compounds such as benzalkonium chloride, cetyltrimethylammonium bromide and cetylpyridinium chloride.

In one embodiment, the aqueous suspensions and dispersions described herein remain in a homogenous state for at least 4 hours. In one embodiment, an aqueous suspension is re-suspended into a homogenous suspension by physical agitation lasting less than 1 minute. In still another embodiment, no agitation is necessary to maintain a homogeneous aqueous dispersion.

An aerosol formulation for nasal administration is generally an aqueous solution designed to be administered to the nasal passages in drops or sprays. Nasal solutions may be similar to nasal secretions in that they are generally isotonic and slightly buffered to maintain a pH of about 5.5 to about 6.5, although pH values outside of this range may additionally be used. Antimicrobial agents or preservatives may also be included in the formulation.

### Kit

Disclosed herein, in some embodiments, are kits for using the engineered polypeptide, the engineered polynucleotide, the system, the pharmaceutical composition described herein. In some embodiments, the kits disclosed herein may be used to treat a disease or condition in a subject. In some embodiments, the kits comprise an assemblage of materials or components apart from the engineered polypeptide, the engineered polynucleotide, the system, the pharmaceutical composition.

In some embodiments, the kit described herein comprises instructions for administering the engineered polypeptide, the engineered polynucleotide, the system, the pharmaceutical composition to the subject to treat a disease or condition in the subject. In some embodiments, the kit further comprises an additional therapeutic agent. In some embodiments, the kit described herein comprises components for forming the first condensate or the second condensate or for determining the formation of the first condensate or the second condensate. In some embodiments, the kit described herein comprises the components for assaying the molecules being modulated by the first condensate or the second condensate. In some embodiments, the kit comprises components for assaying the enzymatic activity associated with the modulated molecules or the concentration of the modulated molecules. In some embodiments, the kit comprises components for performing assays such as western blotting, enzyme-linked immunosorbent assay (ELISA), single-molecular array (Simoa), PCR, and qPCR. The exact nature of the components configured in the kit depends on its intended purpose. For example, some embodiments are configured for the purpose of treating a disease or condition disclosed herein in a subject.

Instructions for use may be included in the kit. In some embodiments, the kit comprises instructions for administering the engineered polypeptide, the engineered polynucleotide, the system, the pharmaceutical composition to a subject in need thereof. Optionally, the kit also contains other useful components such as diluents, buffers, pharmaceutically acceptable carriers, syringes, catheters, applicators, pipetting or measuring tools, bandaging materials or other useful paraphernalia. The materials or components assembled in the kit may be provided to the practitioner stored in any convenient and suitable ways that preserve their operability and utility. For example, the components may be in dissolved, dehydrated, or lyophilized form; they may be provided at room, refrigerated or frozen temperatures. The components are typically contained in suitable packaging material.

Use of absolute or sequential terms, for example, “will,” “will not,” “shall,” “shall not,” “must,” “must not,” “first,” “initially,” “next,” “subsequently,” “before,” “after,” “lastly,” and “finally,” are not meant to limit scope of the present embodiments disclosed herein but as exemplary.

As used herein, the singular forms “a”. “an” and “the” are intended to include the plural forms as well, unless the context clearly indicates otherwise. Furthermore, to the extent that the terms “including”, “includes”, “having”, “has”, “with”, or variants thereof are used in either the detailed description and/or the claims, such terms are intended to be inclusive in a manner similar to the term “comprising.”

As used herein, the phrases “at least one”, “one or more”, and “and/or” are open-ended expressions that are both conjunctive and disjunctive in operation. For example, each of the expressions “at least one of A, B and C”, “at least one of A, B, or C”, “one or more of A, B, and C”, “one or more of A, B, or C” and “A, B, and/or C” means A alone, B alone, C alone, A and B together, A and C together. B and C together, or A, B and C together.

As used herein, “or” may refer to “and”, “or,” or “and/or” and may be used both exclusively and inclusively. For example, the term “A or B” may refer to “A or B”, “A but not B”, “B but not A”, and “A and B”. In some cases, context may dictate a particular meaning.

Any systems, methods, software, and platforms described herein are modular. Accordingly, terms such as “first” and “second” do not necessarily imply priority, order of importance, or order of acts.

The term “about” when referring to a number or a numerical range means that the number or numerical range referred to is an approximation within experimental variability (or within statistical experimental error), and the number or numerical range may vary from, for example, from 1% to 15% of the stated number or numerical range. In examples, the term “about” refers to ±0% of a stated number or value.

The terms “increased”, “increasing”, or “increase” are used herein to generally mean an increase by a statically significant amount. In some aspects, the terms “increased,” or “increase,” mean an increase of at least 10% as compared to a reference level, for example an increase of at least about 10%, at least about 20%, or at least about 30%, or at least about 40%, or at least about 50%, or at least about 60%, or at least about 70%, or at least about 80%, or at least about 90% or up to and including a 100% increase or any increase between 10-100% as compared to a reference level, standard, or control. Other examples of “increase” include an increase of at least 2-fold, at least 5-fold, at least 10-fold, at least 20-fold, at least 50-fold, at least 100-fold, at least 1000-fold or more as compared to a reference level.

The terms “decreased”, “decreasing”, or “decrease” are used herein generally to mean a decrease by a statistically significant amount. In some aspects, “decreased” or “decrease” means a reduction by at least 10% as compared to a reference level, for example a decrease by at least about 20%, or at least about 30%, or at least about 40%, or at least about 50%, or at least about 60%, or at least about 70%, or at least about 80%, or at least about 90% or up to and including a 100% decrease (e.g., absent level or non-detectable level as compared to a reference level), or any decrease between 10-100% as compared to a reference level. In the context of a marker or symptom, by these terms is meant a statistically significant decrease in such level. The decrease can be, for example, at least 10%, at least 20%, at least 30%, at least 40% or more, and is preferably down to a level accepted as within the range of normal for an individual without a given disease.

The terms “individual” or “subject” are used interchangeably and encompass mammals. Non-limiting examples of mammal include any member of the mammalian class: humans, non human primates such as chimpanzees, and other apes and monkey species; farm animals such as cattle, horses, sheep, goats, swine; domestic animals such as rabbits, dogs, and cats; laboratory animals including rodents such as rats, mice and guinea pigs, and the like. The mammal may be a human. The term “animal” as used herein comprises human beings and non human animals. In one embodiment, a “non-human animal” is a mammal, for example a rodent such as rat or a mouse. A “patient,” as used herein refers to a subject that has, or has been diagnosed with, a disease or a condition described herein.

The term “percent (%) identity,” as used herein, generally refers to the percentage of amino acid (or nucleic acid) residues of a candidate sequence that are identical to the amino acid (or nucleic acid) residues of a reference sequence after aligning the sequences and introducing gaps, if necessary, to achieve the maximum percent identity (e.g., gaps may be introduced in one or both of the candidate and reference sequences for optimal alignment and non-homologous sequences may be disregarded for comparison purposes). Alignment, for purposes of determining percent identity, may be achieved in various ways that are commonly known. Percent identity of two sequences may be calculated by aligning a test sequence with a comparison sequence using BLAST, determining the number of amino acids or nucleotides in the aligned test sequence that are identical to amino acids or nucleotides in the same position of the comparison sequence, and dividing the number of identical amino acids or nucleotides by the number of amino acids or nucleotides in the comparison sequence.

As used herein, the term “in vivo” may be used to describe an event that takes place in an organism such as a subject's body.

As used herein, the term “ex vivo” may be used to describe an event that takes place outside of an organism such as subject's body. An “ex vivo” assay cannot be performed on a subject. Rather, it may be performed upon a sample separate from a subject. Ex vivo may be used to describe an event occurring in an intact cell outside a subject's body.

As used herein, the term “in vitro” may be used to describe an event that takes places contained in a container for holding laboratory reagent such that it is separated from the living biological source organism from which the material is obtained. In vitro assays may encompass cell-based assays in which cells alive or dead are employed. In vitro assays may also encompass a cell-free assay in which no intact cells are employed.

“Treat, “treating,” or “treatment,” as used herein, refers to alleviating or abrogating a disorder, disease, or condition; or one or more of the symptoms associated with the disorder, disease, or condition; or alleviating or eradicating a cause of the disorder, disease, or condition itself. Desirable effects of treatment may include, but are not limited to, preventing occurrence or recurrence of disease, alleviation of symptoms, diminishing any direct or indirect pathological consequences of the disease, preventing metastasis, decreasing the rate of disease progression, amelioration or palliation of the disease state and remission or improved prognosis.

The term “effective amount” and “therapeutically effective amount,” as used interchangeably herein, generally refer to the quantity of a composition, for example a composition comprising immune cells such as lymphocytes (e.g., T lymphocytes and/or NK cells) comprising a system of the present disclosure, that is sufficient to result in a desired activity upon administration to a subject in need thereof. Within the context of the present disclosure, the term “therapeutically effective” refers to that quantity of a composition that is sufficient to delay the manifestation, arrest the progression, relieve or alleviate at least one symptom of a disorder treated by the methods of the present disclosure.

The term “pharmaceutically acceptable carrier,” “pharmaceutically acceptable excipient,” “physiologically acceptable carrier,” or “physiologically acceptable excipient” refers to a pharmaceutically-acceptable material, composition, or vehicle such as a liquid or solid filler, diluent, excipient, solvent, or encapsulating material. A component may be “pharmaceutically acceptable” in the sense of being compatible with the other ingredients of a pharmaceutical composition. It may also be suitable for use in contact with the tissue or organ of humans and animals without excessive toxicity, irritation, allergic response, immunogenicity, or other problems or complications, commensurate with a reasonable benefit/risk ratio.

As used herein, the term “administration,” “administering” and variants thereof means introducing a composition or agent into a subject and includes concurrent and sequential introduction of a composition or agent. The introduction of a composition or agent into a subject is by any suitable route, including orally, pulmonarily, intranasally, parenterally (intravenously, intramuscularly, intraperitoneally, or subcutaneously), rectally, intralymphatically, or topically. Administration includes self-administration and administration by another. A suitable route of administration allows the composition or the agent to perform its intended function. For example, if a suitable route is intravenous, the composition is administered by introducing the composition or agent into a vein of the subject. Administration may be carried out by any suitable route. In some embodiments, the administering is intravenous administration. In some embodiments, the administering is pulmonary administration. In some embodiments, the administering is inhalation.

While preferred embodiments of the present invention have been shown and described herein, it will be obvious to those skilled in the art that such embodiments are provided by way of example only. It is not intended that the invention be limited by the specific examples provided within the specification. While the invention has been described with reference to the aforementioned specification, the descriptions and illustrations of the embodiments herein are not meant to be construed in a limiting sense. Numerous variations, changes, and substitutions will now occur to those skilled in the art without departing from the invention. Furthermore, it shall be understood that all aspects of the invention are not limited to the specific depictions, configurations or relative proportions set forth herein which depend upon a variety of conditions and variables. It should be understood that various alternatives to the embodiments of the invention described herein may be employed in practicing the invention. It is therefore contemplated that the invention shall also cover any such alternatives, modifications, variations or equivalents. It is intended that the following claims define the scope of the invention and that methods and structures within the scope of these claims and their equivalents be covered thereby.

### EXAMPLES

The following illustrative examples are representative of embodiments of the stimulation, systems, and methods described herein and are not meant to be limiting in any way.

### Example 1. Condensate Shifting Mediated by Engineered Polypeptide

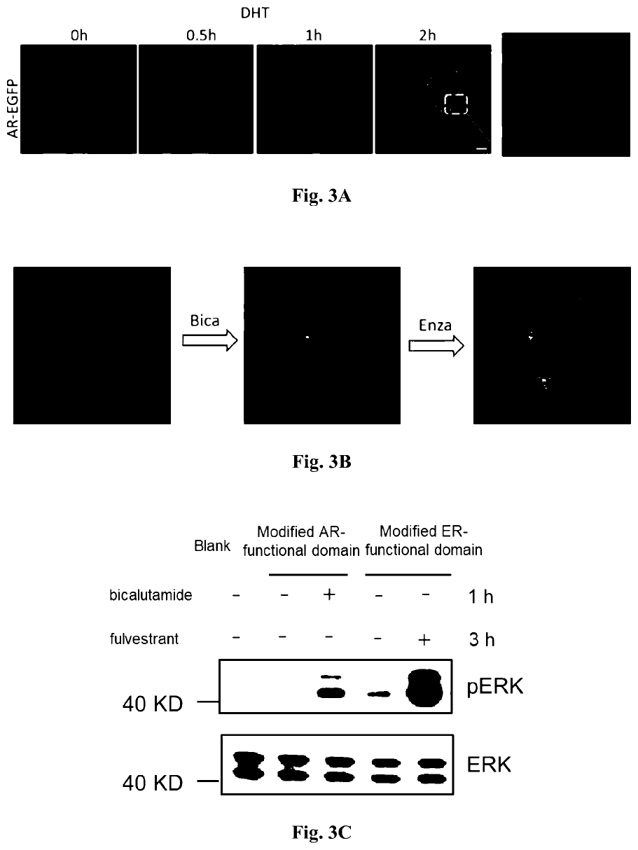

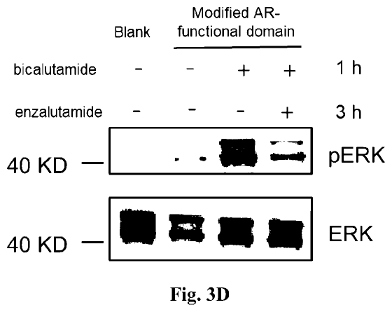

Engineered polypeptide comprising AR or ER condensate shifting domain described herein was furthered fused with a reporter (GFP) and transduced into a cell for monitoring the formation of the first condensate or shifting of the first condensate to the second condensate. Briefly, 293T cells (ATCC) were transfected with AR (SEQ ID NO: 1)-(Δ617-633)-W742L plasmid using Lipofectamine 3000 reagent for 48 hours before taking the pictures (left picture in FIG. 3B) of the cells with Leica THUNDER Imager imaging platform, then, taken pictures (middle picture in FIG. 3B) after adding bicalutamide to the cells for 6 hours, and, taken pictures (right picture in FIG. 3B) after adding enzalutamide to the cells for 6 hours. Engineered polypeptide comprising AR or ER condensate shifting domain described herein was furthered fused with a reporter (GFP) and a functional domain, and then transduced into a cell for testing the signaling cascade change upon formation of the first condensate. Briefly, 293T cells (ATCC) were transfected with AR (SEQ ID NO: 1)-(Δ44-163,226-347,560-670)-W742L-GFP-functional domain plasmid or ER (SEQ ID NO: 5)-(Δ250-274)-L379R-GFP-functional domain using Lipofectamine 3000 reagent for 24 hours, and then were harvested and detected of downstream signaling protein phosphorylation level by western blot, after adding bicalutamide or fulvestrant separately to the cells for 1 hours (FIG. 3C). Engineered polypeptide comprising AR condensate shifting domain described herein was furthered fused with a reporter (GFP) and a functional domain, and then transduced into a cell for testing the signaling cascade change, upon formation of the first condensate or shifting of the first condensate to the second condensate. Briefly, 293T cells (ATCC) were transfected with AR (SEQ ID NO: 1)-(Δ44-163,226-347,560-670)-W742L-GFP-functional domain plasmid using Lipofectamine 3000 reagent for 24 hours, and then were harvest and detected of downstream signaling protein phosphorylation level by western blot, after adding bicalutamide to the cells for 1 hours or adding bicalutamide to the cells for 1 hours and then enzalutamide to the cells for 3 hours in order (FIG. 3D).

While the foregoing disclosure has been described in some detail for purposes of clarity and understanding, it will be clear to one skilled in the art from a reading of this disclosure that various changes in form and detail can be made without departing from the true scope of the disclosure. For example, all the techniques and apparatus described above can be used in various combinations. All publications, patents, patent applications, and/or other documents cited in this application are incorporated by reference in their entirety for all purposes to the same extent as if each individual publication, patent, patent application, and/or other document were individually and separately indicated to be incorporated by reference for all purposes.

1. An engineered polypeptide comprising:
a functional domain; and
a condensate shifting domain, said condensate shifting domain comprises an amino acid sequence of a nuclear receptor or fragment thereof.

2. The engineered polypeptide of claim 1, wherein the engineered polypeptide forms a first condensate within a cell by the engineered polypeptide operatively contacting with at least one additional condensate shifting domain.

3. The engineered polypeptide of claim 1, the engineered polypeptide, upon contacted with a condensate modulator, forms a first condensate within a cell by the engineered polypeptide operatively contacting with at least one additional condensate shifting domain.

4. The engineered polypeptide of claim 2 or 3, wherein the first condensate mediates liquid-liquid phase separation (LLPS).

5. The engineered polypeptide of claim 2 or 3, wherein the first condensate comprises a liquid condensate.

6. The engineered polypeptide of claim 2 or 3, wherein the first condensate allows exchange of molecules between inside and outside of the first condensate.

7. The engineered polypeptide of claim 2 or 3, wherein the first condensate allows movement of molecules inside the first condensate.

8. The engineered polypeptide of claim 3, wherein the first condensate is reversed after removal of the condensate modulator.

9. The engineered polypeptide of claim 2 or 3, wherein the first condensate is reversed when the engineered polypeptide is contacted with a second condensate modulator.

10. The engineered polypeptide of claim 2 or 3 wherein the first condensate comprises a liquid condensate or a liquid-like condensate.

11. The engineered polypeptide of any one of previous claims, wherein the first condensate is shifted to a second condensate when the engineered polypeptide is contacted with a third condensate modulator.

12. The engineered polypeptide of claim 11, wherein the second condensate is reversed back to the first condensate after removal of the third condensate modulator.

13. The engineered polypeptide of claim 11, wherein the second condensate is reversed back to the first condensate when the engineered polypeptide is contacted with a fourth condensate modulator.

14. The engineered polypeptide of claim 11, wherein the second condensate comprises a solid-like condensate or a semi-solid-like condensate.

15. The engineered polypeptide of claim 11, wherein the second condensate comprises a gel-like condensate.

16. The engineered polypeptide of claim 11, wherein the second condensate inhibits exchange of molecules between inside and outside of the second condensate.

17. The engineered polypeptide of claim 11, wherein the second condensate inhibits movement of molecules inside the second condensate.

18. The engineered polypeptide of any one of previous claims, wherein the nuclear receptor or fragment thereof comprises an androgen receptor (AR) or a fragment thereof.

19. The engineered polypeptide of claim 18, wherein the nuclear receptor or fragment thereof comprises an amino acid sequence that is at least 70% identical to SEQ ID NO: 1.

20. The engineered polypeptide of claim 18, wherein the nuclear receptor or fragment thereof comprises an amino acid sequence that is at least 70% identical to SEQ ID NO: 2.

21. The engineered polypeptide of claim 18, wherein the nuclear receptor or fragment thereof comprises an amino acid sequence that is at least 70% identical to SEQ ID NO: 4.

22. The engineered polypeptide of claim 18, wherein the nuclear receptor or fragment thereof does not comprise an amino acid sequence that is at least 70% identical to SEQ ID NO: 3.

23. The engineered polypeptide of claim 18, wherein the nuclear receptor or fragment thereof does not comprise an amino acid sequence that is at least 70% identical to SEQ ID NO: 9.

24. The engineered polypeptide of any one of claims 1-17, wherein the nuclear receptor or fragment thereof comprises an estrogen receptor (ER) or a fragment thereof.

25. The engineered polypeptide of any one of claims 1-17, wherein the nuclear receptor or fragment thereof comprises an amino acid sequence that is at least 70% identical to SEQ ID NO: 5.

26. The engineered polypeptide of any one of claims 1-17, wherein the nuclear receptor or fragment thereof comprises an amino acid sequence that is at least 70% identical to SEQ ID NO: 6.

27. The engineered polypeptide of any one of claims 1-17, wherein the nuclear receptor or fragment thereof comprises an amino acid sequence that is at least 70% identical to SEQ ID NO: 8.

28. The engineered polypeptide of any one of claims 1-17, wherein the nuclear receptor or fragment thereof comprises an amino acid sequence that is at least 70% identical to SEQ ID NO: 7.

29. The engineered polypeptide of claim 18, wherein the nuclear receptor or fragment thereof does not comprise an amino acid sequence that is at least 70% identical to SEQ ID NO: 10.

30. The engineered polypeptide of any one of previous claims, wherein the condensate shifting domain comprises at least one modification.

31. The engineered polypeptide of any one of previous claims, wherein the condensate shifting domain comprises at least one modification compared to any one of SEQ ID NOs: 1-10.

32. The engineered polypeptide of claim 30 or 31, wherein the at least one modification decreases binding between the engineered polypeptide and a nucleic acid by at least 70%, at least 80%, at least 90%, at least 95%, or at least 99% compared to binding between a comparable polypeptide without the at least one modification and the nucleic acid.

33. The engineered polypeptide of claim 30 or 31, wherein the at least one modification abolishes binding between the engineered polypeptide and a nucleic acid.

34. The engineered polypeptide of claim 30 or 31, wherein the at least one modification decreases binding between the engineered polypeptide and a ligand by at least 70%, at least 80%, at least 90%, at least 95%, or at least 99% compared to binding between a comparable polypeptide without the at least one modification and the ligand.

35. The engineered polypeptide of claim 30 or 31, wherein the at least one modification abolishes binding between the engineered polypeptide and the ligand.

36. The engineered polypeptide of claim 34 or 35, wherein the ligand is an endogenous ligand.

37. The engineered polypeptide of claim 34 or 35, wherein the ligand is an exogenous ligand.

38. The engineered polypeptide of claim 30 or 31, wherein the at least one modification comprises at least one truncation.

39. The engineered polypeptide of claim 38, wherein the at least one truncation comprises deletion of a nucleic acid binding domain or a fragment thereof.

40. The engineered polypeptide of claim 30 or 31, wherein the at least one modification comprises at least one amino acid substitution.

41. The engineered polypeptide of claim 30 or 31, wherein the at least one modification comprises at least one truncation and at least one amino acid substitution.

42. The engineered polypeptide of claim 31, wherein the at least one modification comprises Δ574D mutation or W742C mutation compared to SEQ ID NO: 1.

43. The engineered polypeptide of claim 31, wherein the at least one modification comprises Δ574D mutation and W742C mutation compared to SEQ ID NO: 1.

44. The engineered polypeptide of claim 31, wherein the at least one modification comprises Δ574D mutation or W742L mutation compared to SEQ ID NO: 1.

45. The engineered polypeptide of claim 31, wherein the at least one modification comprises Δ574D mutation and W742L mutation compared to SEQ ID NO: 1.

46. The engineered polypeptide of claim 31, wherein the at least one modification comprises K632A mutation; K633A mutation; W742C mutation; or a combination thereof compared to SEQ ID NO: 1.

47. The engineered polypeptide of claim 31, wherein the at least one modification comprises K632A mutation; K633A mutation; and W742C mutation compared to SEQ ID NO: 1.

48. The engineered polypeptide of claim 31, wherein the at least one modification comprises K632A mutation; K633A mutation; W742L mutation; or a combination thereof compared to SEQ ID NO: 1.

49. The engineered polypeptide of claim 31, wherein the at least one modification comprises K632A mutation; K633A mutation; and W742L mutation compared to SEQ ID NO: 1.

50. The engineered polypeptide of claim 31, wherein the at least one modification comprises truncation of amino acid residues 617-633 or W742C mutation compared to SEQ ID NO: 1.

51. The engineered polypeptide of claim 31, wherein the at least one modification comprises truncation of amino acid residues 617-633 and W742C mutation compared to SEQ ID NO: 1.

52. The engineered polypeptide of claim 31, wherein the at least one modification comprises truncation of amino acid residues 617-633 or W742L mutation compared to SEQ ID NO: 1.

53. The engineered polypeptide of claim 31, wherein the at least one modification comprises truncation of amino acid residues 617-633 and W742L mutation compared to SEQ ID NO: 1.

54. The engineered polypeptide of claim 31, wherein the at least one modification comprises truncation of amino acid residues 44-163; truncation of amino acid residues 226-347; truncation of amino acid residues 560-670; W742L mutation; or a combination thereof compared to SEQ ID NO: 1.

55. The engineered polypeptide of claim 31, wherein the at least one modification comprises truncation of amino acid residues 44-163; truncation of amino acid residues 226-347: truncation of amino acid residues 560-670; and W742L mutation compared to SEQ ID NO: 1.

56. The engineered polypeptide of claim 31, wherein the at least one modification comprises truncation of amino acid residues 1-293; truncation of amino acid residues 556-666; W742C mutation; or a combination thereof compared to SEQ ID NO: 1.

57. The engineered polypeptide of claim 31, wherein the at least one modification comprises truncation of amino acid residues 1-293; truncation of amino acid residues 556-666, and W742C mutation compared to SEQ ID NO: 1.

58. The engineered polypeptide of claim 31, wherein the at least one modification comprises truncation of amino acid residues 1-293; truncation of amino acid residues 556-666; W742L mutation; or a combination thereof compared to SEQ ID NO: 1.

59. The engineered polypeptide of claim 31, wherein the at least one modification comprises truncation of amino acid residues 1-293; truncation of amino acid residues 556-666; and W742L mutation compared to SEQ ID NO: 1.

60. The engineered polypeptide of claim 31, wherein the at least one modification comprises truncation of amino acid residues 44-163; truncation of amino acid residues 226-347; truncation of amino acid residues 560-670; W742L mutation; or a combination thereof compared to SEQ ID NO: 1.

61. The engineered polypeptide of claim 31, wherein the at least one modification comprises truncation of amino acid residues 44-163; truncation of amino acid residues 226-347; truncation of amino acid residues 560-670; and W742L mutation compared to SEQ ID NO: 1.

62. The engineered polypeptide of claim 31, wherein the at least one medication comprises Δ574D mutation; W742C mutation; W742L mutation; K632A mutation; K633A mutation; truncation of amino acid residues 617-633; truncation of amino acid residues 44-163: truncation of amino acid residues 226-347; truncation of amino acid residues 560-670; truncation of amino acid residues 1-293; truncation of amino acid residues 556-666; or a combination thereof compared to SEQ ID NO: 1.

63. The engineered polypeptide of claim 31, wherein the at least one modification comprises K210A mutation; R211E mutation; L379R mutation; G521R mutation; or a combination thereof compared to SEQ ID NO: 5.

64. The engineered polypeptide of claim 31, wherein the at least one modification comprises K210A mutation; R211E mutation; L379R mutation; and G521R mutation compared to SEQ ID NO: 5.

65. The engineered polypeptide of claim 31, wherein the at least one modification comprises K210A mutation; R211E mutation; L379R mutation; C400V mutation: M543A mutation; L544A mutation; or a combination thereof compared to SEQ ID NO: 5.

66. The engineered polypeptide of claim 31, wherein the at least one modification comprises K210A mutation; R211E mutation; L379R mutation; C400V mutation: M543A mutation; and L544A mutation compared to SEQ ID NO: 5.

67. The engineered polypeptide of claim 31, wherein the at least one modification comprises truncation of amino acid residues 250-274 or L379R mutation compared to SEQ ID NO: 5.

68. The engineered polypeptide of claim 31, wherein the at least one modification comprises truncation of amino acid residues 250-274 and L379R mutation compared to SEQ ID NO: 5.

69. The engineered polypeptide of claim 31, wherein the at least one modification comprises truncation of amino acid residues 250-274 or G521R mutation compared to SEQ ID NO: 5.

70. The engineered polypeptide of claim 31, wherein the at least one modification comprises truncation of amino acid residues 250-274 and G521R mutation compared to SEQ ID NO: 5.

71. The engineered polypeptide of claim 31, wherein the at least one modification comprises truncation of amino acid residues 250-274; C400V mutation; M543A mutation; L544A mutation; or a combination thereof compared to SEQ ID NO: 5.

72. The engineered polypeptide of claim 31, wherein the at least one modification comprises truncation of amino acid residues 250-274; C400V mutation; M543A mutation; and L544A mutation compared to SEQ ID NO: 5.

73. The engineered polypeptide of claim 31, wherein the at least one modification comprises K210A mutation; R211E mutation; L379R mutation; G521R mutation; C400V mutation; M543A mutation; L544A mutation; truncation of amino acid residues 250-274; or a combination thereof compared to SEQ ID NO: 5.

74. The engineered polypeptide of claim 31, wherein the at least one modification comprises truncation of amino acid residues 44-163; truncation of amino acid residues 226-347; truncation of amino acid residues 560-670: or a combination thereof compared to SEQ ID NO: 9.

75. The engineered polypeptide of claim 31, wherein the at least one modification comprises truncation of amino acid residues 44-163; truncation of amino acid residues 226-347; and truncation of amino acid residues 560-670 compared to SEQ ID NO: 9.

76. The engineered polypeptide of claim 31, wherein the at least one modification comprises truncation of amino acid residues 250-274 compared to SEQ ID NO: 10.

77. The engineered polypeptide of any one of previous claims, wherein the condensate modulator comprises a functional domain ligand.

78. The engineered polypeptide of claim 77, wherein the functional domain ligand comprises a functional domain agonist.

79. The engineered polypeptide of claim 77, wherein the functional domain ligand comprises a functional domain antagonist.

80. The engineered polypeptide of claim 79, wherein the functional domain ligand comprises a functional domain inverse agonist.

81. The engineered polypeptide of any one of previous claims, wherein the condensate modulator comprises a nuclear receptor ligand.

82. The engineered polypeptide of claim 81, wherein the condensate modulator comprises a nuclear receptor agonist, a nuclear receptor antagonist, or a nuclear receptor inverse agonist.

83. The engineered polypeptide of any one of claims 1-76, wherein the condensate modulator comprises an androgen receptor agonist, an androgen receptor antagonist, or an androgen receptor inverse agonist.

84. The engineered polypeptide of any one of claims 1-76, wherein the condensate modulator comprises an androgen receptor ligand comprising bicalutamide.

85. The engineered polypeptide of any one of claims 1-76, wherein the condensate modulator comprises an estrogen receptor agonist, an estrogen receptor antagonist, or an estrogen receptor inverse agonist.

86. The engineered polypeptide of any one of claims 1-76, wherein the condensate modulator comprises an estrogen receptor ligand comprising tamoxifen.

87. The engineered polypeptide of claim 9, 10, or 12, wherein the second condensate modulator, the third condensate modulator, or the fourth condensate modulator comprises a nuclear receptor ligand.

88. The engineered polypeptide of claim 9, 10, or 12, wherein the second condensate modulator, the third condensate modulator, or the fourth condensate modulator comprises a nuclear receptor agonist, a nuclear receptor antagonist, or a nuclear receptor inverse agonist.

89. The engineered polypeptide of claim 9, 10, or 12, wherein the second condensate modulator, the third condensate modulator, or the fourth condensate modulator comprises an androgen receptor agonist, an androgen receptor antagonist, or an androgen receptor inverse agonist.

90. The engineered polypeptide of claim 9, 10, or 12, wherein the second condensate modulator, the third condensate modulator, or the fourth condensate modulator comprises enzalutamide.

91. The engineered polypeptide of claim 90, wherein the second condensate modulator comprises enzalutamide.

92. The engineered polypeptide of claim 9, 10, or 12, wherein the second condensate modulator, the third condensate modulator, or the fourth condensate modulator comprises an estrogen receptor agonist, an estrogen receptor antagonist, or an estrogen receptor inverse agonist.

93. The engineered polypeptide of claim 9, 10, or 12, wherein the second condensate modulator, the third condensate modulator, or the fourth condensate modulator comprises fulvestrant.

94. The engineered polypeptide of claim 93, wherein the third condensate modulator comprises fulvestrant.

95. The engineered polypeptide of any one of previous claims, wherein the engineered polypeptide forms the first condensate or the second condensate in cytoplasm of a cell.

96. The engineered polypeptide of any one of previous claims, wherein the engineered polypeptide forms the first condensate or the second condensate in cytoplasmic membrane of a cell.

97. The engineered polypeptide of any one of claims 1-94, wherein the engineered polypeptide forms the first condensate or the second condensate in an organelle of a cell.

98. The engineered polypeptide of any one of claims 1-94, wherein the engineered polypeptide forms the first condensate or the second condensate in nucleus of a cell.

99. The engineered polypeptide of any one of previous claims, wherein the functional domain comprises a kinase domain.

100. The engineered polypeptide of any one of claims 1-98, wherein the functional domain comprises a phosphatase domain.

101. The engineered polypeptide of any one of claims 1-98, wherein the functional domain comprises a transactivation domain.

102. The engineered polypeptide of any one of claims 1-98, wherein the functional domain comprises a DNA binding domain.

103. The engineered polypeptide of any one of claims 1-98, wherein the functional domain comprises a metabolic enzyme domain.

104. The engineered polypeptide of any one of claims 1-98, wherein the functional domain comprises a receptor.

105. The engineered polypeptide of claim 104, wherein the receptor comprises a CAR or fragment thereof.

106. The engineered polypeptide of claim 104, wherein the receptor comprises a TCR or fragment thereof.

107. An engineered polynucleotide encoding the engineered polypeptide of any one of previous claims.

108. The engineered polynucleotide of claim 107 comprises a vector.

109. The engineered polynucleotide of claim 108, wherein the vector comprises a viral vector.

110. A cell comprising the engineered polypeptide or the engineered polynucleotide of any one of previous claims.

111. A system comprising:
an engineered polypeptide comprising a functional domain and a condensate shifting domain, said condensate shifting domain comprises a amino acid sequence of a nuclear receptor or fragment thereof; and
a condensate modulator,
wherein the engineered polypeptide, upon contacting with the condensate modulator, forms a first condensate comprising the engineered polypeptide operatively contacted with at least one other condensate shifting domain.

112. The system of claim 111, further comprises a second condensate modulator, a third condensate modulator, or a fourth condensate modulator.

113. A pharmaceutical composition comprising the engineered polypeptide, the engineered polynucleotide, the cell, or the system of any one of previous claims.

114. The pharmaceutical composition of claim 113, wherein the pharmaceutical composition is formulated for administering intrathecally, intraocularly, intravitreally, retinally, intravenously, intramuscularly, intraventricularly, intracerebrally, intracerebellarly, intracerebroventricularly, intraperenchymally, subcutaneously, subretinally, suprachoroidally, intratumorally, pulmonarily, endotracheally, intraperitoneally, intravesically, intravaginally, intrarectally, orally, sublingually, transdermally, by inhalation, by inhaled nebulized form, by intraluminal-GI route, or a combination thereof to a subject in need thereof.

115. The pharmaceutical composition of claim 113, wherein the pharmaceutical composition comprises at least one excipient.

116. The pharmaceutical composition of claim 113, wherein the pharmaceutical composition comprises at least one additional active ingredient.

117. A method of forming a first condensate in a cell, said method comprising contacting the cell with the engineered polypeptide, the engineered polynucleotide, or the system of any one of previous claims, wherein the first condensate is formed when the engineered polypeptide is operatively contacted with at least one other condensate shifting domain.

118. The method of claim 117, the engineered polypeptide forms a first condensate within a cell by the engineered polypeptide operatively contacting with at least one additional condensate shifting domain.

119. The method of claim 117, the engineered polypeptide, upon contacted with a condensate modulator, forms a first condensate within a cell by the engineered polypeptide operatively contacting with at least one additional condensate shifting domain.

120. The method of claim 118 or 119, wherein the first condensate mediates liquid-liquid phase separation (LLPS).

121. The method of claim 118 or 119, wherein the first condensate comprises a liquid condensate.

122. The method of claim 118 or 119, wherein the first condensate allows exchange of molecules between inside and outside of the first condensate.

123. The method of claim 118 or 119, wherein the first condensate allows movement of molecules inside the first condensate.

124. The method of claim 119, wherein the first condensate is reversed after removal of the condensate modulator.

125. The method of claim 119, wherein the first condensate is reversed when the engineered polypeptide is contacted with a second condensate modulator.

126. The method of any one of previous claims, wherein the first condensate is shifted to a second condensate when the engineered polypeptide is contacted with a third condensate modulator.

127. The method of claim 126, wherein the second condensate is reversed back to the first condensate after removal of the third condensate modulator.

128. The method of claim 127, wherein the second condensate is reversed back to the first condensate when the engineered polypeptide is contacted with a fourth condensate modulator.

129. The method of claim 127, wherein the second condensate comprises a solid-like condensate.

130. The method of claim 127, wherein the second condensate comprises a semi-solid-like condensate.

131. The method of claim 127, wherein the second condensate comprises a gel-like condensate.

132. The method of claim 127, wherein the second condensate inhibits exchange of molecules between inside and outside of the second condensate.

133. The method of claim 127, wherein the second condensate inhibits movement of molecules inside the second condensate.

134. A method for increasing enzymatic activity in a cell, said method comprising contacting the engineered polypeptide or the engineered polynucleotide of any one of previous claims with a condensate modulator for forming a first condensate when the engineered polypeptide is operatively contacted with at least one other condensate shifting domain, wherein the first condensate enrich local enzyme concentration.

135. A method for increasing enzymatic activity in a cell, said method comprising contacting the engineered polypeptide or the engineered polynucleotide of any one of previous claims with a condensate modulator for forming a first condensate when the engineered polypeptide is operatively contacted with at least one other condensate shifting domain, wherein the first condensate brings two or more molecules of the enzymatic activity to close proximity, thereby increases enzymatic activity.

136. The method of claim 135, wherein the two or more molecules comprise at least one enzyme.

137. The method of claim 135, wherein the two or more molecules comprise at least one biomolecule.

138. The method of claim 135, wherein the two or more molecules comprise at least one nucleic acid.

139. The method of claim 135, wherein the at least one nucleic acid comprises a DNA, a RNA, or a combination thereof.

140. The method of claim 135, wherein the two or more molecules comprise at least one protein.

141. The method of claim 140, wherein the at least one protein comprises a transcription factor.

142. The method of claim 135, wherein the two or more molecules comprise at least one polysaccharide.

143. The method of claim 135, wherein the two or more molecules comprises at least one lipid.

144. The method of claim 135, wherein the two or more molecules comprise at least one metabolite.

145. A method for modulating concentration of at least one molecule in a cell, said method comprising contacting the cell with the engineered polypeptide or the engineered polynucleotide of any one of previous claims, wherein a first condensate is formed when the engineered polypeptide, upon contacted with a condensate modulator, is operatively contacted with at least one other condensate shifting domain, wherein a concentration of the at least one molecule inside the first condensate is different to a concentration of the at least one molecule outside the first condensate.

146. The method of claim 145, wherein the concentration of the at least one molecule inside the first condensate is increased relatively to the concentration of the at least one molecule outside the first condensate.

147. The method of claim 146, wherein the concentration is increased by at least 70%, at least 80%, at least 90%, at least 95%, or at least 99%.

148. The method of claim 147, wherein the concentration of the at least one molecule inside the first condensate is decreased relatively to the concentration of the at least one molecule outside the first condensate.

149. The method of claim 148, wherein the concentration is decreased by at least 70%, at least 80%, at least 90%, at least 95%, or at least 99%.

150. The method of any one claims 117-149, wherein the first condensate is at least 10% more viscous, at least 20% more viscous, at least 30% more viscous, at least 40% more viscous, at least 50% more viscous, at least 60% more viscous, at least 70% more viscous, at least 80% more viscous, at least 90% more viscous, or at least 99% more viscous compared to a comparable first condensate formed by a comparable polypeptide without the at least one modification.

151. The method of any one claims 117-149, wherein the first condensate is at least 10% less viscous, at least 20% less viscous, at least 30% less viscous, at least 40% less viscous, at least 50% less viscous, at least 60% less viscous, at least 70% less viscous, at least 80% less viscous, at least 90% less viscous, or at least 99% less viscous compared to a comparable first condensate formed by a comparable polypeptide without the at least one modification.

152. The method of any one claims 117-151, wherein the first condensate comprises at least 10% more surface tension, at least 20% more surface tension, at least 30% more surface tension, at least 40% more surface tension, at least 50% more surface tension, at least 60% more surface tension, at least 70% more surface tension, at least 80% more surface tension, at least 90% more surface tension, or at least 99% more surface tension compared to a comparable first condensate formed by a comparable polypeptide without the at least one modification.

153. The method of any one claims 117-151, wherein the first condensate comprises at least 10% less surface tension, at least 20% less surface tension, at least 30% less surface tension, at least 40% less surface tension, at least 50% less surface tension, at least 60% less surface tension, at least 70% less surface tension, at least 80% less surface tension, at least 90% less surface tension, or at least 99% less surface tension compared to a comparable first condensate formed by a comparable polypeptide without the at least one modification.

154. The method of any one claims 117-153, wherein the first condensate comprises at least 10% more turbidity, at least 20% more turbidity, at least 30% more turbidity, at least 40% more turbidity, at least 50% more turbidity, at least 60% more turbidity, at least 70% more turbidity, at least 80% more turbidity, at least 90% more turbidity, or at least 99% more turbidity compared to a comparable first condensate formed by a comparable polypeptide without the at least one modification.

155. The method of any one claims 117-153, wherein the first condensate comprises at least 10% less turbidity, at least 20% less turbidity, at least 30% less turbidity, at least 40% less turbidity, at least 50% less turbidity, at least 60% less turbidity, at least 70% less turbidity, at least 80% less turbidity, at least 90% less turbidity, or at least 99% less turbidity compared to a comparable first condensate formed by a comparable polypeptide without the at least one modification.

156. The method of any one claims 117-144, wherein the first condensate increases a rate of exchange of molecule between inside and outside of the first condensate by at least 10%, at least 20%, at least 30%, at least 40%, at least 50%, at least 60%, at least 70%, at least 80%, at least 90%, or at least 99% compared to a rate of exchange of molecule between inside and outside of a comparable first condensate formed by a comparable polypeptide without the at least one modification.

157. The method of any one claims 117-155, wherein the first condensate decreases a rate of exchange of molecule between inside and outside of the first condensate by at least 10%, at least 20%, at least 30%, at least 40%, at least 50%, at least 60%, at least 70%, at least 80%, at least 90%, or at least 99% compared to a rate of exchange of molecule between inside and outside of a comparable first condensate formed by a comparable polypeptide without the at least one modification.

158. The method of any one claims 117-157, wherein the first condensate is at least 10%, at least 20%, at least 30%, at least 40%, at least 50%, at least 60%, at least 70%, at least 80%, at least 90%, or at least 99% more likely to accumulate in cytoplasm of a cell compared to a comparable first condensate formed by a comparable polypeptide without the at least one modification.

159. A method for treating a disease or condition in a subject in need thereof, comprising administering the engineered polypeptide, the engineered polynucleotide, the cell, or the system of any one of previous claims to a subject.

160. The method of claim 159, wherein the engineered polypeptide, the engineered polynucleotide, the cell, or the system of any one of previous claims forms a first condensate, a second condensate, a third condensate, or a combination thereof for treating the disease or condition in the subject.
